(19) United States
(12) Patent Application Publication
(10) Pub. No.: US 2025/0152750 A1
(43) Pub. Date: May 15, 2025

(54) CYANINE DYE NANOPARTICLES FOR SENESCENCE IMAGING

(71) Applicant: Cancer Research Technology Limited, London (GB)

(72) Inventors: Ljiljana Fruk, Cambridge (GB); Daniel Munoz Espin, Cambridge (GB); Andrew Baker, Cambridge (GB)

(21) Appl. No.: 18/838,447

(22) PCT Filed: Feb. 21, 2023
(86) PCT No.: PCT/GB23/50386

§ 371 Date: Aug. 14, 2024

(30) Foreign Application Priority Data
Feb. 21, 2022 (GB) 2202336.0

(51) Int. Cl. A61K 49/00; A61P 35/00; G01N 21/64

(52) U.S. Cl. CPC A61K 49/0032; A61K 49/0093; A61P 35/00; G01N 21/6428; G01N 2021/6439

(57) ABSTRACT

The present invention relates to a process for preparing a nanoparticle composition comprising cyanine dye nanoparticles. The present invention also relates to said nanoparticles and their uses for detecting senescent cells and determining senescent burden.

The present invention relates to a process for preparing a nanoparticle composition comprising cyanine dye nanoparticles. The present invention also relates to said nanoparticles and their uses for detecting senescent cells and determining senescent burden.

### BACKGROUND

Senescence is a cellular damage and stress response that triggers a halt in division and the secretion of proinflammatory cytokines and chemokines to aid in the repair of tissue. However, in instances where senescent cells are not cleared by the immune system or upon persistent cellular damage, organ and organismal dysfunction can occur. Senescent cells have been associated with the ageing process as well as the progression of pathological manifestations of multiple diseases such as fibrotic disorders (e.g. pulmonary and kidney fibrosis), cardiovascular diseases (e.g. atherosclerosis), neurological disorders (Alzheimer's and Parkinson disease), skeletomuscular disorders (osteoarthritis and sarcopenia), diabetes 1 and 2, and many different cancer types. The presence of senescent cells has been demonstrated in all stages of cancer development, including initiation, although these cells can commonly play different roles, depending on the context. For example, in pre-cancerous lesions such as prostatic intraepithelial neoplasia (PIN) or lung adenomas, they are believed to halt the progression into full blown cancer. However, these cells can also create pro-tumourigenic environments and can contribute to relapse/formation of metastatic cancer cells in tumours by modulating the microenvironment. Such roles, commonly related to the persistence of uncleared senescent cells, have also been demonstrated in cancer tissue after chemotherapy treatment where the accumulation of senescent cells leads to development of a pro-tumourigenic environment, often leading to the cancer relapse after treatment. Furthermore, the unspecific nature of current chemotherapeutic strategies means that senescent cells can also be formed outside the initial tumor site, which might lead to undesired side effects and secondary cancer formation, even later in life. Thus, senescent cells are becoming an important target not only for treatment of age-related diseases but also to reduce harmful side effects of chemotherapies and prevent the formation of secondary cancers post treatment, and even as a cancer preventative strategy in groups at high cancer risk.

Reports have shown that in some types of cancers, Idiopathic pulmonary fibrosis and Alzheimer's disease, the elimination of senescent cells can lead to more desirable treatment outcomes and health spans. For example, senescent cells targeted by a combination of Dasatinib and quercetin led to an improvement in physical function of patients as well as a reduction in senescent and inflammatory markers. Moreover, it has been shown in mouse models of aging that targeting and eliminating senescent cells improved lifespan by 30% while at the same time reducing the mortality hazard by 65%. In spite of this, there remains a number of challenges burdening therapeutic interventions at the senescent cell level, such as their identification in vivo using available medical imaging and diagnostic strategies.

Currently there are several probes available for identification of senescent cells, and the most widely used ones are based on monitoring the increased activity of the lysosomal β-galactosidase enzyme. Most of these probes employ dyes which indicate the presence of senescent cells through colorimetric change or increased fluorescence, although other means of identification, such as a PET probe containing [¹⁸F] label (FPyGal), have also been reported, but still at a preclinical stage. Alternative probes which do not rely on the senescence-associated β-galactosidase activity employ mitochondrial targeting using the near infrared (NIR) cyanine dye, CyBC9, which can detect senescent mesenchymal stromal cells. Despite these advances, the currently available dye-probes are based on readout strategies suitable for in vitro studies and tissue staining but cannot be readily applied to an in vivo or human setting. Due to the heterogeneity of senescence, there is also a general lack of suitable targets that are specific enough to be able to detect the presence of senescent cells in a complex tissue environment.

There is therefore a need for stable probe systems which are suitable for in vivo detection and monitoring of senescent cells as well as imaging senescence burden in a biological sample.

The present invention was devised with the foregoing in mind.

### BRIEF SUMMARY OF THE DISCLOSURE

According to a first aspect, the present invention provides a process for preparing a nanoparticle composition comprising cyanine dye nanoparticles dispersed in an aqueous medium, the process comprising:
(i) providing an aqueous solution of the cyanine dye;
(ii) maintaining the aqueous solution of the cyanine dye at a temperature of between 15° C. to 85° C., preferably with agitation, to allow the cyanine dye molecules to aggregate and form nanoparticles;
(iii) collecting and purifying the resultant nanoparticles present in the solution;

wherein step (iii) comprises at least one separation step wherein the cyanine dye nanoparticles formed in step (ii) are separated from the aqueous solution and re-suspended or dispersed in a different aqueous medium. Suitably, step (iii) comprises at least one centrifugation step wherein the solution is centrifuged to form a pellet of the cyanine dye nanoparticles, removing the supernatant, optionally washing the pellet, and re-suspending the pellet in a different aqueous medium.

According to a second aspect, the present invention provides cyanine dye nanoparticles obtainable by/obtained by/directly obtained by a process defined herein.

According to a third aspect, the present invention provides a nanoparticle composition obtainable by/obtained by/directly obtained by a process defined herein.

According to a fourth aspect, the present invention provides a nanoparticle composition comprising cyanine dye nanoparticles as defined herein dispersed in an aqueous medium.

According to a fifth aspect, the present invention provides a nanoparticle composition comprising cyanine dye nanoparticles dispersed in an aqueous medium, wherein:
(i) the composition is substantially free of cyanine dye in a non-aggregated form in the solution; and/or
(ii) the composition is stable for greater than 12 hours.

According to a sixth aspect, the present invention provides a nanoparticle composition consisting essentially of, or consisting of, cyanine dye nanoparticles dispersed in an aqueous medium.

According to a seventh aspect, the present invention provides an imaging agent comprising cyanine dye nanoparticles defined herein, or a nanoparticle composition defined herein.

According to an eighth aspect, the present invention provides cyanine dye nanoparticles defined herein, or a nanoparticle composition defined herein, or an imaging agent defined herein, for use in:
(i) detecting senescent cells in a biological sample in vitro, ex vivo or in vivo; or
(ii) determining the senescent burden of a cell population in a biological sample in vitro, ex vivo or in vivo.

According to a ninth aspect, the present invention provides a use of cyanine dye nanoparticles defined herein, or a nanoparticle composition defined herein, or an imaging agent defined herein, for:
(i) detecting senescent cells in a biological sample in vitro, ex vivo or in vivo; or
(ii) determining the senescent burden of a cell population in a biological sample in vitro, ex vivo or in vivo.

According to a tenth aspect, the present invention provides an imaging method for:
(i) detecting senescent cells in a biological sample in vitro, ex vivo or in vivo; or
(ii) determining the senescent burden of a cell population in a biological sample in vitro, ex vivo or in vivo;
   the method comprising contacting cyanine dye nanoparticles as defined herein, or a nanoparticle composition as defined herein, or an imaging agent as defined herein, to the biological sample and imaging the sample to determine the distribution of the nanoparticles and identify senescent cells in the sample.

### DETAILED DESCRIPTION

### Definitions

Unless otherwise stated, the following terms used in the specification and claims have the following meanings set out below.

The term “aryl” or “aromatic” as used herein means an aromatic ring system comprising 6, 7, 8, 9 or 10 ring carbon atoms. Aryl is often phenyl but may be a polycyclic ring system, having two or more rings, at least one of which is aromatic. This term includes reference to groups such as phenyl, naphthyl and the like.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom.

The term “carbocyclyl”, “carbocyclic” or “carbocycle” means a non-aromatic saturated or partially saturated monocyclic, or a fused, bridged, or spiro bicyclic carbocyclic ring system(s). Monocyclic carbocyclic rings contain from about 3 to 12 (suitably from 3 to 7) ring atoms. Bicyclic carbocycles contain from 7 to 17 carbon atoms in the rings, suitably 7 to 12 carbon atoms, in the rings. Bicyclic carbocyclic rings may be fused, spiro, or bridged ring systems.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems.

The term “substituted” as used herein in reference to a moiety means that one, two, three, four or more positions on the moiety are substituted. Preferably, “substituted” as used herein in reference to a moiety means that 1, 2 or 3, of the hydrogen atoms in said moiety are replaced independently of each other by the corresponding number of substituents. Even more preferred, “substituted” as used herein in reference to a moiety means that 1 or 2, of the hydrogen atoms in said moiety are replaced independently of each other by the corresponding number of substituents.

It will, of course, be understood that substituents are only at positions where they are chemically possible, the person skilled in the art being able to decide (either experimentally or theoretically) without inappropriate effort whether a particular substitution is possible.

Throughout the entirety of the description and claims of this specification, where subject matter is described herein using the term “comprise” (or “comprises” or “comprising”), the same subject matter instead described using the term “consist of” (or “consists of” or “consisting of”) or “consist essentially of” (or “consists essentially of” or “consisting essentially of”) is also contemplated.

Furthermore, throughout the entirety of the description and claims of this specification, the singular encompasses the plural unless the context otherwise requires. In particular, where the indefinite article is used, the specification is to be understood as contemplating plurality as well as singularity, unless the context requires otherwise.

When a component is described as “consisting essentially of” a subsequently recited material, it is to be understood that the component concerned may contain 70% or more, 75% or more, 80% or more, 85% or more, 90% or more, 95% or more, 96% or more, 97% or more, 98% or more, 99% or more, 99.5% or more, of the subsequently recited material.

Features described in conjunction with a particular aspect, embodiment or example of the present invention are to be understood to be applicable to any other aspect, embodiment or example described herein unless clearly incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The present invention is not restricted to the details of any of the specific embodiments recited herein. The present invention extends to any novel one, or any novel combination, of the features disclosed in this specification (including any accompanying claims, abstract and drawings), or to any novel one, or any novel combination, of the steps of any method or process so disclosed.

### Preparation of the Nanoparticles

In accordance with a first aspect, the present invention provides a process for preparing a nanoparticle composition comprising cyanine dye nanoparticles dispersed in an aqueous medium, the process comprising:
(i) providing an aqueous solution of the cyanine dye;
(ii) maintaining the aqueous solution of the cyanine dye at a temperature of between 15° C. to 85° C., preferably with agitation, to allow the cyanine dye molecules to aggregate and form nanoparticles;
(iii) collecting and purifying the resultant nanoparticles present in the solution;
   wherein step (iii) comprises at least one separation step wherein the cyanine dye nanoparticles formed in step (ii) are separated from the aqueous solution and re-suspended or dispersed in a different aqueous medium.

Cyanine dyes are a known term in the art and will be understood to relate to compounds which typically comprise a polymethine chain attached to at least one organic moiety such as a heterocycle, heteroaryl, aryl and/or a carbocycle. It will, of course, be understood that these are merely exemplary organic moieties, and the invention is not limited in this regard. Rather, the term cyanine dye will be understood to encompass compounds which typically comprise a polymethine chain (of any length) attached to at least one organic moiety. For example, it may be that the polymethine chain is attached to at least one organic moiety such as pyrrole, imidazole, thiazole, pyridine, quinoline, indole and/or benzothiazole, any of which may be optionally substituted. It may also be that the polymethine chain is attached to at least one organic moiety such as an aryl, a heteroaryl, a carbocycle or a heterocycle, any of which may be optionally substituted. The at least one organic moiety may be attached to the polymethine chain in any manner and on any atom of the polymethine chain. Typically, the at least one organic moiety is a terminal group on the polymethine chain. When the at least one organic moiety is a terminal group on the polymethine chain, the cyanine dyes can be classified as closed chain cyanines (two ring organic moieties (e.g., heterocycles) are present at the terminal ends of the polymethine chain), hemicyanines (one ring organic moiety and one non-ring organic moiety are present at the terminal ends of the polymethine chain), streptocyanines (two non-ring organic moieties are present at the terminal ends of the polymethine chain) and merocyanines (one amino group and one carbonyl group are present at the terminal ends of the polymethine chain). Suitably, the cyanine dye is a closed chain cyanine, a hemicyanine, a streptocyanine or a merocyanine.

The cyanine dye may comprise a polymethine chain of any length. Suitably, the cyanine dye comprises a polymethine chain of 1-20 methine units. More suitably, the cyanine dye comprises a polymethine chain of 1-10 methine units. Yet more suitably, the cyanine dye comprises a polymethine chain of 1-5 methine units. Alternatively, the cyanine dye does not comprise a methine unit and the organic moieties are directly linked. Such a cyanine dye is classified as an apocyanine. Suitably, the cyanine dye is an apocyanine.

Suitably, the cyanine dye is selected from the group consisting of indocyanine green (ICG), IR-140, IR-820, IR-806, IR783, IR780, Cy7, and Cy7.5. In an embodiment, the cyanine dye is indocyanine green (ICG). Furthermore, the cyanine dye may be modified by any suitable means and all modified cyanine dyes are encompassed within the scope of the present invention. For example, it may be that any of the cyanine dyes discussed herein may be modified by any suitable process with any suitable moiety (i.e., polyamine modification). In an embodiment, the cyanine dye is a modified cyanine dye. In an embodiment, the cyanine dye is modified indocyanine green (ICG). It will be appreciated that any of the cyanine dyes discussed herein, including modified cyanine dyes, will be suitable in the formation of cyanine dye nanoparticles. For example, it may be that any of the cyanine dyes discussed herein can aggregate to form J-aggregates (i.e., the cyanine dye nanoparticles aggregate to form J-aggregate nanoparticles).

In embodiments where the cyanine dye is ICG, the cyanine dye nanoparticles may be further characterised by a characteristic J-absorption band at 895 nm.

The cyanine dyes described herein may be in the form of nanoparticles. The cyanine dye nanoparticles of the present invention may have a particle size of less than 1000 nm. The cyanine dye nanoparticles may have a particle size of less than 800 nm. Suitably, the cyanine dye nanoparticles have a particle size of less than 600 nm. More suitably, the cyanine dye nanoparticles have a particle size of less than 400 nm. Even more suitably, the cyanine dye nanoparticles have a particle size of less than 200 nm. Yet even more suitably, the cyanine dye nanoparticles have a particle size of less than 100 nm.

In step (i) and/or step (ii), the pH of the aqueous solution may be maintained within an appropriate range. Suitably the pH of the aqueous solution in step (i) and/or step (ii) is within the range of 2 to 9. More suitably, the pH of the aqueous solution in step (i) and/or step (ii) is within the range of 2 to 8. Even more suitably, the pH of the aqueous solution in step (i) and/or step (ii) is within the range of 2 to 7. Yet even more suitably, the pH of the aqueous solution in step (i) and/or step (ii) is within the range of 2 to 6.

Step (i) of the process for preparing a nanoparticle composition comprising cyanine dye nanoparticles dispersed in an aqueous medium comprises providing an aqueous solution of the cyanine dye. Suitably, the concentration of cyanine dye in the aqueous solution in step (i) is within the range of 0.1 mM to 10 mM. More suitably, the concentration of cyanine dye in the aqueous solution in step (i) is within the range of 0.5 mM to 7.5 mM. Even more suitably, the concentration of cyanine dye in the aqueous solution in step (i) is within the range of 0.75 mM to 5 mM. Yet even more suitably, the concentration of cyanine dye in the aqueous solution in step (i) is within the range of 1.0 mM to 3 mM.

In step (i), the cyanine dye is provided in an aqueous solution. The aqueous solution of the cyanine dye may comprise water. Suitably, the aqueous solution of the cyanine dye is a solution of the cyanine dye in water.

In step (ii) the aqueous solution of the cyanine dye is maintained at a temperature of between 15° C. to 85° C. It will be appreciated that the cyanine dye may be maintained at any temperature within this range for any appropriate period of time. Suitably, in step (ii), the solution is heated to a temperature within the range of 40° C. to 85° C., optionally for 0.5 to 48 hours. More suitably, in step (ii), the solution is heated to a temperature within the range of 45° C. to 85° C., optionally for 0.5 to 48 hours. Yet more suitably, in step (ii), the solution is heated to a temperature within the range of 55° C. to 75° C., optionally for 0.5 to 48 hours. Yet even more suitably, in step (ii), the solution is maintained within the stated temperature range defined herein for 0.5 to 24 hours, and optionally for 0.5 to 6 hours or 0.5 to 3 hours.

In step (ii), the process may further comprise monitoring the formation of nanoparticles, optionally by monitoring the depletion of the non-aggregated cyanine dye from the aqueous solution and/or the formation of the nanoparticles.

In step (iii), the at least one separation step separates the cyanine dye nanoparticles formed in step (ii) from the aqueous solution to remove any non-aggregated cyanine dye from the aqueous medium. The separation step may be carried out multiple times to ensure the cyanine dye nanoparticles are substantially free from an non-aggregated cyanine dye.

In step (iii), any suitable separation technique that isolates the nanoparticles formed in step (ii) from the remainder of the aqueous solution (and any non-aggregated cyanine dye) may be used. Suitable techniques include centrifugation, filtration and/or dialysis. It will be appreciated that any suitable method of dialysing the solution of cyanine dye nanoparticles may be used. It will also be appreciated that any suitable method of filtering the solution of cyanine dye nanoparticles may also be used.

In step (iii), the collection and purification of the cyanine dye nanoparticles may comprise two or more, or three or more, separation (e.g. centrifugation, filtration and/or dialysis) steps.

Suitably, step (iii) comprises at least one centrifugation step wherein the solution is centrifuged to form a pellet of the cyanine dye nanoparticles, removing the supernatant, optionally washing the pellet, and re-suspending the pellet in a different aqueous medium.

In embodiments where the separation technique in step (iii) includes at least one centrifugation step, it will be appreciated that any suitable centrifugal force may be used, provided that the centrifugal force is sufficient enough to form a pellet of the cyanine dye nanoparticles. Suitably, the centrifugal force of step (iii) is 20,000×g (RCF) to 50,000×g (RCF). More suitably, the centrifugal force of step (iii) is 25,000×g (RCF) to 45,000×g (RCF). Yet more suitably, the centrifugal force of step (iii) is 27,500×g (RCF) to 42,500×g (RCF). In an embodiment, the centrifugal force of step (iii) is 30,000×g (RCF) to 40,000×g (RCF). Suitably, the centrifugation step is performed at 10,000 rpm to 25,000 rpm. More suitably, the centrifugation step is performed at 12,500 rpm to 22,500 rpm. Even more suitably, the centrifugation step is performed at 15,000 rpm to 20,000 rpm. In an embodiment, the centrifugation step is performed at 17,000 rpm to 18,000 rpm. In such embodiments, the collection and purification of the cyanine dye nanoparticles may further comprise additional steps of dialysing the solution of cyanine dye nanoparticles and/or filtering the solution of cyanine dye nanoparticles.

### Nanoparticles, Nanoparticle Compositions and Imaging Agents Thereof

In accordance with a second aspect, the present invention provides cyanine dye nanoparticles obtainable by/obtained by/directly obtained by a process as defined herein.

In accordance with a third aspect, the present invention provides a nanoparticle composition obtainable by/obtained by/directly obtained by a process as defined herein.

In accordance with a fourth aspect, the present invention provides a nanoparticle composition comprising cyanine dye nanoparticles as defined herein dispersed in an aqueous medium.

In accordance with a fifth aspect, the present invention provides a nanoparticle composition comprising cyanine dye nanoparticles dispersed in an aqueous medium, wherein:
(i) the composition is substantially free of cyanine dye in a non-aggregated form in the solution; and/or
(ii) the composition is stable for greater than 12 hours.

Suitably, the cyanine dye nanoparticles of the fifth aspect of the present invention are obtainable by/obtained by/directly obtained by a process as defined herein.

It will be appreciated that, in step (i), wherein the composition is substantially free of cyanine dye in a non-aggregated form in the solution, this will be understood to mean that the nanoparticle composition comprises 50% or less, 40% or less, 30% or less, 20% or less, 10% or less, 5% or less, 4% or less, 3% or less, 2% or less, 1% or less, or 0% of cyanine dye in a non-aggregated form in the solution.

In accordance with a sixth aspect, the present invention provides a nanoparticle composition consisting essentially of, or consisting of, cyanine dye nanoparticles dispersed in an aqueous medium.

The nanoparticle composition of the sixth aspect may contain 70% or more, 75% or more, 80% or more, 85% or more, 90% or more, 95% or more, 96% or more, 97% or more, 98% or more, 99% or more, or 99.5% or more, of cyanine dye nanoparticles dispersed in an aqueous medium.

Suitably, the cyanine dye nanoparticles of the sixth aspect of the present invention are obtainable by/obtained by/directly obtained by a process as defined herein.

It will be understood that the following paragraphs are applicable to the second, third, fourth, fifth, sixth and seventh aspects of the present invention.

The cyanine dye may be a closed chain cyanine, a hemicyanine, a streptocyanine or a merocyanine.

The cyanine dye may comprise a polymethine chain of any length. Suitably, the cyanine dye comprises a polymethine chain of 1-20 methine units. More suitably, the cyanine dye comprises a polymethine chain of 1-10 methine units. Yet more suitably, the cyanine dye comprises a polymethine chain of 1-5 methine units. Alternatively, the cyanine dye does not comprise a methine unit and the organic moieties are directly linked. Such a cyanine dye is classified as an apocyanine. Suitably, the cyanine dye is an apocyanine.

Suitably, the cyanine dye is selected from the group consisting of indocyanine green (ICG), IR-140, IR-820, IR-806, IR783, IR780, Cy7, and Cy7.5. In an embodiment, the cyanine dye is indocyanine green (ICG). Furthermore, the cyanine dye may be modified by any suitable means and all modified cyanine dyes are encompassed within the scope of the present invention. For example, it may be that any of the cyanine dyes discussed herein may be modified by any suitable process with any suitable moiety (i.e., polyamine modification). In an embodiment, the cyanine dye is a modified cyanine dye. In an embodiment, the cyanine dye is modified indocyanine green (ICG). It will be appreciated that any of the cyanine dyes discussed herein, including modified cyanine dyes, will be suitable in the formation of cyanine dye nanoparticles. For example, it may be that any of the cyanine dyes discussed herein can aggregate to form J-aggregates (i.e., the cyanine dye nanoparticles aggregate to form J-aggregate nanoparticles).

In embodiments where the cyanine dye is ICG, the cyanine dye nanoparticles may be further characterised by a characteristic J-absorption band at 895 nm.

The cyanine dye nanoparticles may have a particle size of less than 1000 nm. The cyanine dye nanoparticles may have a particle size of less than 800 nm. Suitably, the cyanine dye nanoparticles have a particle size of less than 600 nm. More suitably, the cyanine dye nanoparticles have a particle size of less than 400 nm. Even more suitably, the cyanine dye nanoparticles have a particle size of less than 200 nm. Yet even more suitably, the cyanine dye nanoparticles have a particle size of less than 100 nm.

In accordance with a seventh aspect, the present invention provides an imaging agent comprising cyanine dye nanoparticles, or a nanoparticle composition as defined herein, and one or more pharmaceutically acceptable excipients.

The cyanine dye nanoparticles, or a nanoparticle composition as defined herein, and one or more pharmaceutically acceptable excipients can be used as an imaging agent for fluorescent imaging or for photoacoustic imaging. It will be appreciated that the term “imaging agent” will be understood to mean any agent which is suitable for imaging a sample. In an embodiment, the imaging agent is a contrast agent. It will be further appreciated that the term “imaging agent” and “contrast agent” can be used interchangeably throughout all aspects of the present invention. The contrast agent can absorb near infrared light and emit fluorescence or an acoustic wave. It will be understood that the contrast agent can display a difference in contrast between a biological sample or molecule that is subject to observation.

### Use of the Nanoparticles

In accordance with an eighth aspect, the present invention provides cyanine dye nanoparticles, or a nanoparticle composition, or an imaging agent as defined herein for use in:
(i) detecting senescent cells in a biological sample in vitro, ex vivo or in vivo; or
(ii) determining the senescent burden of a cell population in a biological sample in vitro, ex vivo or in vivo.

Furthermore, in accordance with a ninth aspect, the present invention provides use of cyanine dye nanoparticles, or a nanoparticle composition, or an imaging agent as defined herein, for:
(i) detecting senescent cells in a biological sample in vitro, ex vivo or in vivo; or
(ii) determining the senescent burden of a cell population in a biological sample in vitro, ex vivo or in vivo.

The term “senescent burden” used in relation to any aspect of the present invention is a known term in the art and will be understood to mean the proportion of senescent cells/cellular senescence in a sample.

It will be understood that the following paragraphs are applicable to the eighth and ninth aspects of the present invention.

The cyanine dye nanoparticles, nanoparticle composition or imaging agent may be used to assess the amount of senescence/the senescent burden in a biological sample (e.g., a cell population, organoid, 3D (e.g., 3D bioprints etc.), tissue sample or body tissue) in vitro, ex vivo or in vivo in response to one or more stimuli, optionally selected from replicative (ageing) stress, oncogene-induction, oxidative stress, therapeutic/drug treatment, radiation exposure, viral or bacterial infection, or other agents or stress factors. It may be that the cyanine dye nanoparticles, nanoparticle composition or imaging agent is used to assess the amount of senescence/the senescent burden in a biological sample before, after or during treatment of the biological sample with one or more senolytics, one or more chemotherapeutic agents and/or radiation therapy. Treatment of the biological sample with one or more senolytics may be combined with one or more additional treatments, such as chemotherapy and/or radiation therapy. It will be understood that the one or more additional treatments, such as chemotherapy and/or radiation therapy may take place before, during or after treatment of the biological sample with one or more senolytics. Therefore, the cyanine dye nanoparticles, nanoparticle composition or imaging agents of the present invention are important in the detection and monitoring of the treatment of diseases and/or disorders wherein the level of senescence/senescent burden is indicative of the severity of the disease and/or disorder. Therefore, the cyanine dye nanoparticles, nanoparticle composition or imaging agents of the present invention can be used for: (i) determining whether treatment with a senolytic, one or more chemotherapeutic agents and/or radiation therapy, is required; and/or (ii) monitoring the effect of a senolytic treatment, chemotherapy and/or radiation therapy, on a disease and/or disorder. The level of senescence/the senescent burden can be indicative of the severity of the disease and/or disorder and the impact of a senolytic treatment, chemotherapeutic agents and/or radiation therapy, can be assessed and monitored with the cyanine dye nanoparticles, nanoparticle composition or imaging agent of the present invention. Thus, the eighth and ninth aspects of the present invention may further comprise the cyanine dye nanoparticles, nanoparticle composition or imaging agents of the present invention for use in detecting senescent cells in a biological sample in vitro, ex vivo or in vivo; or determining the senescent burden of a cell population in a biological sample in vitro, ex vivo or in vivo, before, during or after the treatment of the biological sample with the one or more senolytics, chemotherapeutic agents and/or radiation therapy. Illustrative examples of senolytic drugs include dasatinib, quercetin, fisetin and navitoclax. Suitable examples of chemotherapeutic agents are well known in the art.

Suitably, the cyanine dye nanoparticles, nanoparticle composition or imaging agent is used for:
(i) detecting the presence of senescent cells or determining the senescent burden of:
   a pre-malignant lesion or tumour, or
   an age-related disorder (e.g., chronic disorders, including but not restricted to, cardiovascular diseases, fibrosis, neurological disorders, Type 1 & 2 diabetes, sarcopenia, osteoarthritis, osteoporosis, inflammatory diseases, chronic obstructive pulmonary disease (COPD), infarction, aneurysm, cataracts, post-infarction tissues, etc.),
      and optionally wherein a treatment (e.g. with one or more senolytics, one or more chemotherapeutic agents and/or radiation therapy) is determined based on the detection of the presence of senescent cells and/or the determination of the senescent burden;
(ii) detecting the presence of senescent cells or determining the senescent burden of a pre-malignant lesion, tumour or age-related disorder before, during or after treatment;
(iii) detecting the presence of senescent cells or determining the senescent burden of a biological sample or tissue to assess and/or monitor biological aging, developmental senescence (e.g., in physiological processes), tissue dysfunction, inflammation, viral or bacterial infection, the accumulation of senescent cells (e.g., physiological or pathological senescence);
(iv) detecting the presence of senescent cells or determining the senescent burden of a biological sample or tissue before, during or after treatment with one or more senolytics, one or more chemotherapeutic agents and/or radiation therapy.

Suitably, the cyanine dye nanoparticles, nanoparticle composition or imaging agent is used for diagnostic, prognostic and/or predictive applications.

Suitably, the presence of senescent cells is determined by magnetic resonance imaging (MRI), positron emission tomography (PET), near infrared II (NIR-II) imaging, shortwave infrared (SWIR) imaging, two-photon microscopy, fluorescence imaging or photoacoustic imaging or a combination thereof. In an embodiment, the presence of the senescent cells is determined by fluorescence imaging or photoacoustic imaging.

It will be appreciated that the term “developmental senescence” used anywhere herein may refer to predictable and reproducible senescence in response to developmental cues in one or more physiological processes (e.g., tissue remodelling in embryonic development). Indeed, senescence has previously been shown to occur during mammalian embryonic development at multiple locations, such as the mesonephros, the endolymphatic sac of the inner ear, closing neural tube, and developing limbs. In such processes, the loss of senescence can in fact lead to developmental abnormalities. Therefore, it will be appreciated that detecting the presence of senescent cells or determining the senescent burden of a biological sample or tissue in “developmental senescence” will encompass detecting the loss of senescent cells or determining the loss of senescent burden of a biological sample or tissue. Suitably, detecting the presence of senescent cells or determining the senescent burden of a biological sample or tissue to assess and/or monitor developmental senescence is conducted in vitro, ex vivo or in vivo. More suitably, detecting the presence of senescent cells or determining the senescent burden of a biological sample or tissue to assess and/or monitor developmental senescence is conducted ex vivo.

Imaging method

In accordance with a tenth aspect, the present invention provides an imaging method for:
(i) detecting senescent cells in a biological sample in vitro, ex vivo or in vivo; or
(ii) determining the senescent burden of a cell population in a biological sample in vitro, ex vivo or in vivo;
   the method comprising contacting cyanine dye nanoparticles as defined herein, or a nanoparticle composition as defined herein, or an imaging agent as defined herein, to the biological sample and imaging the sample to determine the distribution of the nanoparticles and identify senescent cells in the sample.

Imaging the biological sample may comprise application of at least one imaging technique selected from the group consisting of: magnetic resonance imaging (MRI), positron emission tomography (PET), near infrared II (NIR-II) imaging, shortwave infrared (SWIR) imaging, two-photon microscopy, photoacoustic imaging and fluorescent imaging or any combination thereof.

The image may be analysed to assess the amount of senescence in a biological sample (e.g., a cell population, organoid, 3D (e.g., 3D bioprints etc.), or tissue sample) in vitro, ex vivo or in vivo in response to one or more stimuli, optionally selected from replicative (ageing) stress, oncogene-induction, oxidative stress, therapeutic/drug treatment, radiation exposure, viral or bacterial infection, or other agents or stress factors.

Suitably, the imaging method is a method of:
(i) detecting the presence of senescent cells or determining the senescent burden of:
   a pre-malignant lesion or tumour, or
   an age-related disorder (e.g., chronic disorders, including but not restricted to, cardiovascular diseases, fibrosis, neurological disorders, Type 1 & 2 diabetes, sarcopenia, osteoarthritis, osteoporosis, inflammatory diseases, COPD, infarction, aneurysm, cataracts, post-infarction tissues),
      and optionally wherein a treatment (e.g. with one or more senolytics, one or more chemotherapeutic agents and/or radiation therapy) is determined based on the detection of the presence of senescent cells and/or the determination of the senescent burden;
(ii) detecting the presence of senescent cells or determining the senescent burden of a pre-malignant lesion, tumour or age-related disorder before, during or after treatment;
(iii) detecting the presence of senescent cells or determining the senescent burden of a biological sample or tissue to assess and/or monitor biological aging, developmental senescence (e.g., in physiological processes), tissue dysfunction, inflammation, viral or bacterial infection, the accumulation of senescent cells (e.g., physiological or pathological senescence);

Suitably, the step of contacting cyanine dye nanoparticles, or a nanoparticle composition, or an imaging agent as defined herein, to the biological sample comprises administering the cyanine dye nanoparticles, nanoparticle composition or imaging agent to a subject, and imaging the subject or a tissue in the subject to determine the distribution of the nanoparticles in vivo and identify any senescent cells in the subject or a tissue in the subject.

The following numbered statements 1 to 67 are not claims, but instead define particular aspects and embodiments of the claimed invention:

1. A process for preparing a nanoparticle composition comprising cyanine dye nanoparticles dispersed in an aqueous medium, the process comprising:
(i) providing an aqueous solution of the cyanine dye;
(ii) maintaining the aqueous solution of the cyanine dye at a temperature of between 15° C. to 85° C., preferably with agitation, to allow the cyanine dye molecules to aggregate and form nanoparticles;
(iii) collecting and purifying the resultant nanoparticles present in the solution; wherein step (iii) comprises at least one separation step wherein the cyanine dye nanoparticles formed in step (ii) are separated from the aqueous solution and re-suspended or dispersed in a different aqueous medium.

2. A process for preparing a nanoparticle composition according to statement 1, wherein the cyanine dye is a closed chain cyanine, a hemicyanine, a streptocyanine, a merocyanine or an apocyanine.

3. A process for preparing a nanoparticle composition according to statement 1 or 2, wherein the cyanine dye comprises a polymethine chain of 1-20 methine units.

4. A process for preparing a nanoparticle composition according to statement 1, 2 or 3, wherein the cyanine dye comprises a polymethine chain of 1-10 methine units.

5. A process for preparing a nanoparticle composition according to any one of the preceding statements, wherein the cyanine dye comprises a polymethine chain of 1-5 methine units.

6. A process for preparing a nanoparticle composition according to any one of the preceding statements, wherein the cyanine dye is selected from the group consisting of indocyanine green (ICG), IR-140, IR-820, IR-806, IR783, IR780, Cy7, and Cy7.5.

7. A process for preparing a nanoparticle composition according to any one of the preceding statements, wherein the cyanine dye is indocyanine green (ICG).

8. A process for preparing a nanoparticle composition according to any one of the preceding statements, wherein the cyanine dye is a modified cyanine dye.

9. A process for preparing a nanoparticle composition according to any one of the preceding statements, wherein the cyanine dye is modified indocyanine green (ICG).

10. A process for preparing a nanoparticle composition according to any one of the preceding statements, wherein the cyanine dye forms J-aggregates.

11. A process for preparing a nanoparticle composition according to any one of the preceding statements, wherein the cyanine dye nanoparticles have a particle size of less than 1000 nm.

12. A process for preparing a nanoparticle composition according to any one of the preceding statements, wherein the cyanine dye nanoparticles have a particle size of less than 800 nm.

13. A process for preparing a nanoparticle composition according to any one of the preceding statements, wherein the cyanine dye nanoparticles have a particle size of less than 600 nm.

14. A process for preparing a nanoparticle composition according to any one of the preceding statements, wherein the cyanine dye nanoparticles have a particle size of less than 400 nm.

15. A process for preparing a nanoparticle composition according to any one of the preceding statements, wherein the cyanine dye nanoparticles have a particle size of less than 200 nm.

16. A process for preparing a nanoparticle composition according to any one of the preceding statements, wherein the cyanine dye nanoparticles have a particle size of less than 100 nm.

17. A process for preparing a nanoparticle composition according to any one of the preceding statements, wherein the pH of the aqueous solution in step (i) and/or step (ii) is within the range of 2 to 9.

18. A process according to any one of the preceding statements, wherein the pH of the aqueous solution in step (i) and/or step (ii) is within the range of 2 to 8.

19. A process according to any one of the preceding statements, wherein the pH of the aqueous solution in step (i) and/or step (ii) is within the range of 2 to 7.

20. A process according to any one of the preceding statements, wherein the pH of the aqueous solution in step (i) and/or step (ii) is within the range of 2 to 6.

21. A process according to any one of the preceding statements, wherein the concentration of cyanine dye in the aqueous solution in step (i) is within the range of 0.1 mM to 10 mM.

22. A process according to any one of the preceding statements, wherein the concentration of cyanine dye in the aqueous solution in step (i) is within the range of 0.5 mM to 7.5 mM.

23. A process according to any one of the preceding statements, wherein the concentration of cyanine dye in the aqueous solution in step (i) is within the range of 0.75 mM to 5 mM.

24. A process according to any one of the preceding statements, wherein the concentration of cyanine dye in the aqueous solution in step (i) is within the range of 1.0 mM to 3 mM.

25. A process according to any one of the preceding statements, wherein the aqueous solution of the cyanine dye comprises water.

26. A process according to any one of the preceding statements, wherein the aqueous solution of the cyanine dye is a solution of the cyanine dye in water.

27. A process according to any one of the preceding statements, wherein, in step (ii), the solution is heated to a temperature within the range of 40° C. to 85° C., optionally for 0.5 to 48 hours.

28. A process according to any one of the preceding statements, wherein, in step (ii), the solution is heated to a temperature within the range of 45° C. to 85° C., optionally for 0.5 to 48 hours.

29. A process according to any one of the preceding statements, wherein, in step (ii), the solution is heated to a temperature within the range of 55° C. to 75° C., optionally for 0.5 to 48 hours.

30. A process according to any one of statements 27 to 29, wherein, in step (ii), the solution is maintained within the stated temperature range for 0.5 to 24 hours, and optionally for 0.5 to 6 hours or 0.5 to 3 hours.

31. A process according to any one of the preceding statements, wherein, in step (ii), the process further comprises monitoring the formation of nanoparticles, optionally by monitoring the depletion of the non-aggregated cyanine dye from the aqueous solution and/or the formation of the nanoparticles.

32. A process according to any one of the preceding statements, wherein, in step (iii), the collection and purification of the cyanine dye nanoparticles comprises one, two or more or three or more centrifugation steps.

33. A process according to any one of the preceding statements, wherein, in step (iii), the collection and purification of the cyanine dye nanoparticles comprises steps of dialysing the solution of cyanine dye nanoparticles and/or filtering the solution of cyanine dye nanoparticles.

34. Cyanine dye nanoparticles obtainable by/obtained by/directly obtained by a process as defined in any one of statements 1 to 33.

35. A nanoparticle composition obtainable by/obtained by/directly obtained by a process as defined in any one of statements 1 to 33.

36. A nanoparticle composition comprising cyanine dye nanoparticles of statement 34 dispersed in an aqueous medium.

37. A nanoparticle composition comprising cyanine dye nanoparticles dispersed in an aqueous medium, wherein:
(i) the composition is substantially free of cyanine dye in a non-aggregated form in the solution; and/or
(ii) the composition is stable for greater than 12 hours.

38. A nanoparticle composition consisting essentially of, or consisting of, cyanine dye nanoparticles dispersed in an aqueous medium.

39. A nanoparticle composition according to statement 37 or statement 38, wherein the cyanine dye nanoparticles are obtainable by/obtained by/directly obtained by a process as defined in any one of statements 1 to 33.

40. Cyanine dye nanoparticles according to statement 34, or a nanoparticle composition according to any of statements 35 to 39, wherein the cyanine dye is a closed chain cyanine, a hemicyanine, a streptocyanine, a merocyanine or an apocyanine.

41. Cyanine dye nanoparticles according to statement 34, or a nanoparticle composition according to any of statements 35 to 39, wherein the cyanine dye comprises a polymethine chain of 1-20 methine units.

42. Cyanine dye nanoparticles according to statement 34, or a nanoparticle composition according to any of statements 35 to 39, wherein the cyanine dye comprises a polymethine chain of 1-10 methine units.

43. Cyanine dye nanoparticles according to statement 34, or a nanoparticle composition according to any of statements 35 to 39, wherein the cyanine dye comprises a polymethine chain of 1-5 methine units.

44. Cyanine dye nanoparticles according to statement 34, or a nanoparticle composition according to any of statements 35 to 39, wherein the cyanine dye is selected from the group consisting of indocyanine green (ICG), IR-140, IR-820, IR-806, IR783, IR780, Cy7, and Cy7.5.

45. Cyanine dye nanoparticles according to statement 34, or a nanoparticle composition according to any of statements 35 to 39, wherein the cyanine dye is indocyanine green (ICG).

46. Cyanine dye nanoparticles according to statement 34, or a nanoparticle composition according to any of statements 35 to 39, wherein the cyanine dye is a modified cyanine dye.

47. Cyanine dye nanoparticles according to statement 34, or a nanoparticle composition according to any of statements 35 to 39, wherein the cyanine dye is modified indocyanine green (ICG).

48. Cyanine dye nanoparticles according to statement 34, or a nanoparticle composition according to any of statements 35 to 39, wherein the cyanine dye forms J-aggregates.

49. Cyanine dye nanoparticles according to statement 34, or a nanoparticle composition according to any of statements 35 to 39, wherein the cyanine dye nanoparticles have a particle size of less than 1000 nm.

50. Cyanine dye nanoparticles according to statement 34, or a nanoparticle composition according to any of statements 35 to 39, wherein the cyanine dye nanoparticles have a particle size of less than 800 nm.

51. Cyanine dye nanoparticles according to statement 34, or a nanoparticle composition according to any of statements 35 to 39, wherein the cyanine dye nanoparticles have a particle size of less than 600 nm.

52. Cyanine dye nanoparticles according to statement 34, or a nanoparticle composition according to any of statements 35 to 39, wherein the cyanine dye nanoparticles have a particle size of less than 400 nm.

53. Cyanine dye nanoparticles according to statement 34, or a nanoparticle composition according to any of statements 35 to 39, wherein the cyanine dye nanoparticles have a particle size of less than 200 nm.

54. Cyanine dye nanoparticles according to statement 34, or a nanoparticle composition according to any of statements 35 to 39, wherein the cyanine dye nanoparticles have a particle size of less than 100 nm.

55. An imaging agent comprising cyanine dye nanoparticles according to statement 34, or a nanoparticle composition according to any of statements 35 to 39, and one or more pharmaceutically acceptable excipients.

56. Cyanine dye nanoparticles according to statement 34, or a nanoparticle composition according to any of statements 35 to 39, or an imaging agent according to statement 55, for use in:
(i) detecting senescent cells in a biological sample in vitro, ex vivo or in vivo; or
(ii) determining the senescent burden of a cell population in a biological sample in vitro, ex vivo or in vivo.

57. Use of cyanine dye nanoparticles according to statement 34, or a nanoparticle composition according to any of statements 35 to 39, or an imaging agent according to statement 55, for:
(i) detecting senescent cells in a biological sample in vitro, ex vivo or in vivo; or
(ii) determining the senescent burden of a cell population in a biological sample in vitro, ex vivo or in vivo.

58. Cyanine dye nanoparticles according to statement 34, or a nanoparticle composition according to any of statements 35 to 39, or an imaging agent according to statement 55 for use according to statement 56; or the use of cyanine dye nanoparticles according to statement 34, or a nanoparticle composition according to any of statements 35 to 39, or an imaging agent according to statement 55, according to statement 57;

wherein the cyanine dye nanoparticles, nanoparticle composition or imaging agent is used to assess the amount of senescence in a biological sample (e.g., a cell population, organoid, 3D (e.g., 3D bioprints etc.), or tissue sample) in vitro, ex vivo or in vivo in response to one or more stimuli, optionally selected from replicative (ageing) stress, oncogene-induction, oxidative stress, therapeutic/drug treatment, radiation exposure, viral or bacterial infection, or other agents or stress factors.

59. Cyanine dye nanoparticles according to statement 34, or a nanoparticle composition according to any of statements 35 to 39, or an imaging agent according to statement 55 for use according to statement 56; or the use of cyanine dye nanoparticles according to statement 34, or a nanoparticle composition according to any of statements 35 to 39, or an imaging agent according to statement 55, according to statement 57;
wherein the cyanine dye nanoparticles, nanoparticle composition or imaging agent is used for:
   (i) detecting the presence of senescent cells or determining the senescent burden of:
      a pre-malignant lesion or tumour, or
      an age-related disorder (e.g., chronic disorders, including but not restricted to, cardiovascular diseases, fibrosis, neurological disorders, Type 1 & 2 diabetes, sarcopenia, osteoarthritis, osteoporosis, inflammatory diseases, COPD, infarction, aneurysm, cataracts, post-infarction tissues, etc.),
         and optionally wherein a treatment (e.g. with one or more senolytics, one or more chemotherapeutic agents and/or radiation therapy) is determined based on the detection of the presence of senescent cells and/or the determination of the senescent burden;
   (ii) detecting the presence of senescent cells or determining the senescent burden of a pre-malignant lesion, tumour or age-related disorder before, during or after treatment;
   (iii) detecting the presence of senescent cells or determining the senescent burden of a biological sample or tissue to assess and/or monitor biological aging, developmental senescence (e.g., in physiological processes), tissue dysfunction, inflammation, viral or bacterial infection, the accumulation of senescent cells (e.g., physiological or pathological senescence);
   (iv) detecting the presence of senescent cells or determining the senescent burden of a biological sample or tissue before, during or after treatment with one or more senolytics, one or more chemotherapeutic agents and/or radiation therapy.

60. Cyanine dye nanoparticles according to statement 34, or a nanoparticle composition according to any of statements 35 to 39, or an imaging agent according to statement 55 for use according to statement 56; or
the use of cyanine dye nanoparticles according to statement 34, or a nanoparticle composition according to any of statements 35 to 39, or an imaging agent according to statement 55, according to statement 57;
wherein the cyanine dye nanoparticles, nanoparticle composition or imaging agent is used for diagnostic, prognostic and/or predictive applications.

61. Cyanine dye nanoparticles according to statement 34, or a nanoparticle composition according to any of statements 35 to 39, or an imaging agent according to statement 55 for use according to statement 56; or
the use of cyanine dye nanoparticles according to statement 34, or a nanoparticle composition according to any of statements 35 to 39, or an imaging agent according to statement 55, according to statement 57;
wherein the presence of the senescent cells is determined by magnetic resonance imaging (MRI), positron emission tomography (PET), near infrared II (NIR-II) imaging, shortwave infrared (SWIR) imaging, two-photon microscopy, fluorescence imaging or photoacoustic imaging or a combination thereof.

62. An imaging method for:
(i) detecting senescent cells in a biological sample in vitro, ex vivo or in vivo; or
(ii) determining the senescent burden of a cell population in a biological sample in vitro, ex vivo or in vivo;
the method comprising contacting cyanine dye nanoparticles according to statement 34, or
   a nanoparticle composition according to any of statements 35 to 39, or an imaging agent according to statement 55, to the biological sample and imaging the sample to determine the distribution of the nanoparticles and identify senescent cells in the sample.

63. An imaging method according to statement 62, wherein imaging the biological sample comprises application of at least one imaging technique selected from the group consisting of: magnetic resonance imaging (MRI), positron emission tomography (PET), near infrared II (NIR-II) imaging, shortwave infrared (SWIR) imaging, two-photon microscopy, photoacoustic imaging and fluorescent imaging or any combination thereof.

64. An imaging method according to statement 62 or 63, wherein the image is analysed to assess the amount of senescence in a biological sample (e.g., a cell population, organoid, 3D (e.g., 3D bioprints etc.), or tissue sample) in vitro, ex vivo or in vivo in response to one or more stimuli, optionally selected from replicative (ageing) stress, oncogene-induction, oxidative stress, therapeutic/drug treatment, radiation exposure, viral or bacterial infection, or other agents or stress factors.

65. An imaging method according to statement 62, 63 or 64, wherein the method is a method of:
(i) detecting the presence of senescent cells or determining the senescent burden of:
   a pre-malignant lesion or tumour, or
   an age-related disorder (e.g., chronic disorders, including but not restricted to, cardiovascular diseases, fibrosis, neurological disorders, Type 1 & 2 diabetes, sarcopenia, osteoarthritis, osteoporosis, inflammatory diseases, COPD, infarction, aneurysm, cataracts, post-infarction tissues, etc),
      and optionally wherein a treatment (e.g. with one or more senolytics, one or more chemotherapeutic agents and/or radiation therapy) is determined based on the detection of the presence of senescent cells and/or the determination of the senescent burden;
(ii) detecting the presence of senescent cells or determining the senescent burden of a pre-malignant lesion, tumour or age-related disorder before, during or after treatment;
(iii) detecting the presence of senescent cells or determining the senescent burden of a biological sample or tissue to assess and/or monitor biological aging, developmental senescence (e.g., in physiological processes), tissue dysfunction, inflammation, viral or bacterial infection, the accumulation of senescent cells (e.g., physiological or pathological senescence);
(iv) detecting the presence of senescent cells or determining the senescent burden of a biological sample or tissue before, during or after treatment with one or more senolytics, one or more chemotherapeutic agents and/or radiation therapy.

66. An imaging method according to any one of statements 62 to 65, wherein the step of contacting cyanine dye nanoparticles according to statement 34, or a nanoparticle composition according to any of statements 35 to 39, or an imaging agent according to statement 55, to the biological sample comprises administering the cyanine dye nanoparticles, nanoparticle composition or imaging agent to a subject, and imaging the subject or a tissue in the subject to determine the distribution of the nanoparticles in vivo and identify any senescent cells in the subject or a tissue in the subject.

67. A process according to statement 32, wherein, the centrifugation step is performed at 10,000 rpm to 25,000 rpm, 12,500 rpm to 22,500 rpm, 15,000 rpm to 20,000 rpm, or 17,000 rpm to 18,000 rpm.

### EXAMPLES—PART A

### Figures

FIG. 1a) shows the reaction mechanism of cyanine dye nanoparticle (CDNP) formation from Indocyanine green (ICG) precursors. In short, an aqueous solution of ICG is heated at 65° C. for 24 hours. After the reaction is complete the resulting solution is centrifuged 3 times and further processed by dialysis and filtration before application.

FIG. 1b) shows characteristic red shift of absorbance upon cyanine dye nanoparticle formation, demonstrating aggregation of ICG.

FIG. 1c) shows a cryo TEM image of the cyanine dye nanoparticles, indicating the average size is 69 nm+−16 nm.

FIG. 1d) shows a demonstration of green cyanine dye nanoparticle accumulation using two models of cellular senescence by using human melanoma (SK-MEL-103) and lung cancer (A549) cells treated with Palbociclib.

FIG. 1e) shows the fluorescence emission of a cyanine dye nanoparticle compared to a monomer of ICG, both of which are excited at 633 nm. Also shown in FIG. 1e) is photoacoustic spectra of an ICG monomer compared to the cyanine dye nanoparticle, demonstrating increased photoacoustic efficiency.

FIG. 1.1a) shows the UV-Vis spectral stability of cyanine dye nanoparticles in a variety of biological media over a 1 week period at intervals of 24 hrs. The upper panels show the spectrum in each case, and the lower panels plot the ratio of absorbance (890 nm compared to 780 nm), demonstrating a negligible change over the 1 week period.

FIG. 1.1b) shows the viability assessment of various concentrations of cyanine dye nanoparticles in control and senescent cells for SK-MEL-103 and A549 cells. No marked toxicity is seen.

FIG. 1.1c) shows UV-Vis spectral changes with ethanol. Cyanine dye nanoparticles can be dissolved in methanol, ethanol, or nonpolar solvents, and their UV-vis spectrums resemble that of ICG. However, this is not the case for its fluorescence, which then has characteristics of both the cyanine dye nanoparticle and ICG monomer, albeit markedly reduced.

FIG. 1.2 shows the NMR and mass spectrum of ICG in deuterated methanol and methanol (i & ii). The mass spectrum shows the characteristic mass at 752 m/z, and the NMR spectrum matches previously published data. Further the UV-Vis spectrum and an image of the starting solution (iii) are shown.

FIG. 1.3 shows the NMR and mass spectrum of the pellet of pure cyanine dye nanoparticles, which contains the characteristic mass of ICG. The NMR matches the reported spectrums in Mindt et al (2018) and Rüttger et al (2019), who identify ICG dimer using mass spectroscopy and NMR. However, this only contains masses associated with ICG (i & ii). The UV-Vis spectrum and an image of cyanine dye nanoparticle is shown as a strong green colour (iii).

FIG. 1.4 shows the NMR and multiple mass spectra of the supernatant obtained after centrifugation (i-iiii). In the mass spectra there were multiple peaks in chromatogram, and none of them contain the characteristic ICG mass (ii-iiii). The UV-Vis spectrum of the resulting solution shows the cyanine dye nanoparticle peak as well as the monomer peak. An image of the solution shows a greyish blue colour.

FIG. 2a) shows the accumulation of cyanine dye nanoparticles in cells evaluated with flow cytometry. This demonstrates an increase in accumulation of senescent cells compared to the controls.

FIG. 2b) shows confocal comparison of SK-MEL-103 control and senescent cells induced by Palbociclib and treated with 50 μg/mL cyanine dye nanoparticles. Confocal quantification of grey values of images are compared.

FIG. 2c) shows confocal comparison of A549 control and senescent cells induced by Palbociclib. Confocal quantification of grey values of images are compared.

FIG. 2d) shows confocal comparison of A549 control and senescent cells induced by cisplatin (CDDP). Confocal quantification of grey values of images are compared.

FIG. 2e) shows confocal comparison of WI-38 control and senescent cells induced by radiation. Confocal quantification of grey values of images are compared.

FIG. 2.2a) shows senescence associated-β-galactosidase (SA-β-Gal) staining. Senescent cells all show enhanced staining.

FIG. 2.2b) shows growth curves of control (black) and senescent (red) cells. All senescent cells show reduced growth capacity compared to proliferative controls. For ER: MEK IMR90 cells, a comparison of SA-β-Gal activity is shown.

FIG. 2.2c) shows Western blots of cellular senescence markers, demonstrating a reduction in phosphorylated retinoblastoma protein (p-RB), increased p21 and p53 levels in senescent cells compared to control cells.

FIG. 3 shows that cyanine dye nanoparticles are up taken by multiple endocytosis routes and accumulate in lysosomes. Accumulation of cyanine dye nanoparticles in control cells and senescent cells in absence (no inhibitor) and presence of endocytosis pathways inhibitors. Quantification of obtained confocal images (control cells are shown in grey). Chloroquine addition causes a more dramatic increase of cyanine dye nanoparticles in control cells compared to senescent. Pitstop 2 and dyngo4a inhibitors both result in a significant inhibition of cyanine dye nanoparticle uptake in senescent cells.

FIG. 3.1a) shows the accumulation of ICG in multiple models of senescence analysed by flow cytometry.

FIG. 4a) shows an overview of an in vivo experiment using untreated and Palbociclib treated SK-MEL-103 xenografts.

FIG. 4b) shows senescence associated β-galactosidase staining of untreated and Palbociclib tumours, demonstrating senescent cells presence in palbociclib treated tumours (green stain).

FIG. 4c) shows how cyanine dye nanoparticles target senescent tumours in comparison to untreated tumours.

FIG. 4d) shows quantification of fluorescence of cyanine dye nanoparticles in untreated and palbociclib treated tumours.

FIG. 4e) shows how ICG demonstrates accumulation in untreated tumours as opposed to senescent tumours.

FIG. 4f) shows quantification of ICG emission in untreated and Palbociclib tumours.

FIG. 4g) shows confocal microscopy of slices of untreated and Palbociclib treated tumours. Palbociclib treated tumours show increased fluorescence in cyanine dye nanoparticle signal, and signal near cells.

FIG. 4.1a) shows confocal comparison of SK-MEL-103 control and senescent cells induced by palbociclib and treated with 50 μg/mL cyanine dye nanoparticles.

FIG. 4.1b) shows confocal comparison of A549 control and senescent cells induced by palbociclib.

FIG. 4.1c) shows confocal comparison of A549 control and senescent cells induced by cisplatin (CDDP).

FIG. 4.1d) shows confocal comparison of WI-38 control and senescent cells induced by radiation.

FIG. 5a) shows an image of tumours and one set of organs in both untreated and Palbociclib treated mice. Kidneys also demonstrate some signal, however not as much as treated tumour. Increased signal in kidneys indicate renal clearance mechanism.

FIG. 5b) shows emission of untreated and Palbociclib treated tumours in the presence of cyanine dye nanoparticles excited in the ICG channel. This demonstrates an insignificant difference in fluorescence.

FIG. 6a) shows an outline of the method of the present invention compared to prior art methods. Addition of centrifugation steps further clean the cyanine dye nanoparticles of the present invention.

FIG. 6b) shows the compounds in the supernatant and product yields in the method of the present invention.

FIG. 7 shows a graphical abstract of cyanine dye nanoparticles (green) targeting senescent cells due to increased endocytosis. Senescent cells shown enlarged and accumulating lysosomes in light blue, control cells shown in beige.

### MATERIALS AND METHODS

### Cyanine Dye Nanoparticle Synthesis

Cyanine dye nanoparticles were prepared in Milli Q deionised water. Aqueous ICG solutions (0.75 mM, 10 mL) were sonicated for 10 min then heated to 65° C. under stirring (500 rpm). The reaction was monitored by a UV-Vis spectrophotometer. Samples were then centrifuged and washed three times at 17,000 rpm (31000×g) at 4° C. for 30 min. The centrifugation step is required to obtain stable and pure nanoparticles. Cyanine dye nanoparticles were purified firstly by dialysis using 1 kDa bag against DI water for two days. The hydrodynamic size of cyanine dye nanoparticles was measured using DLS Zetasizer.

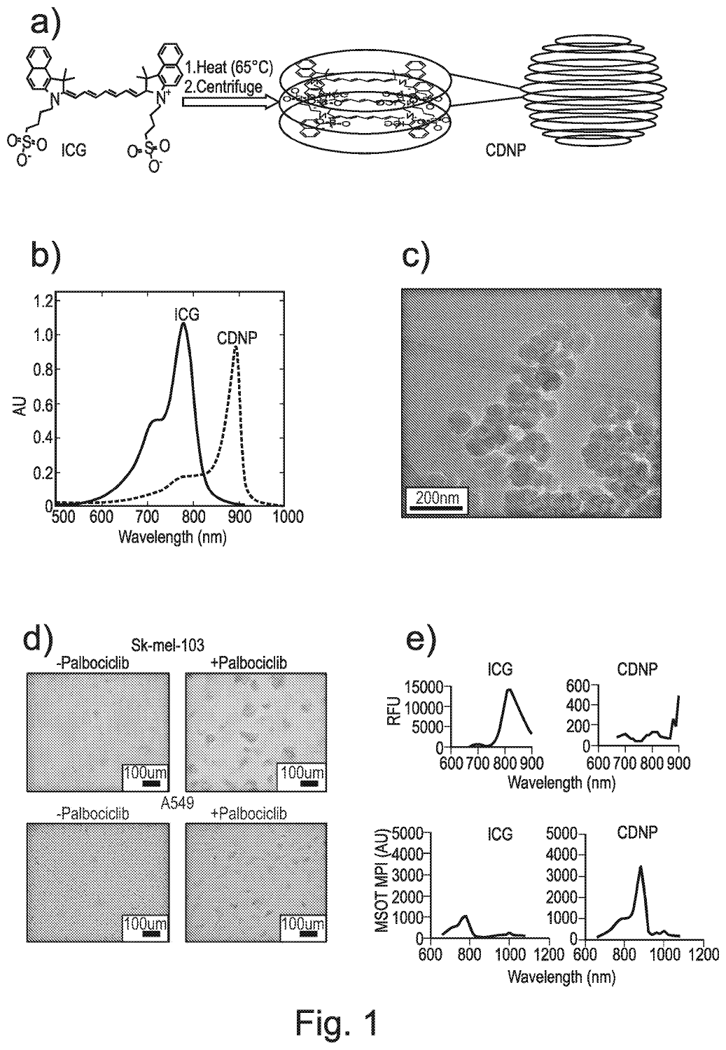

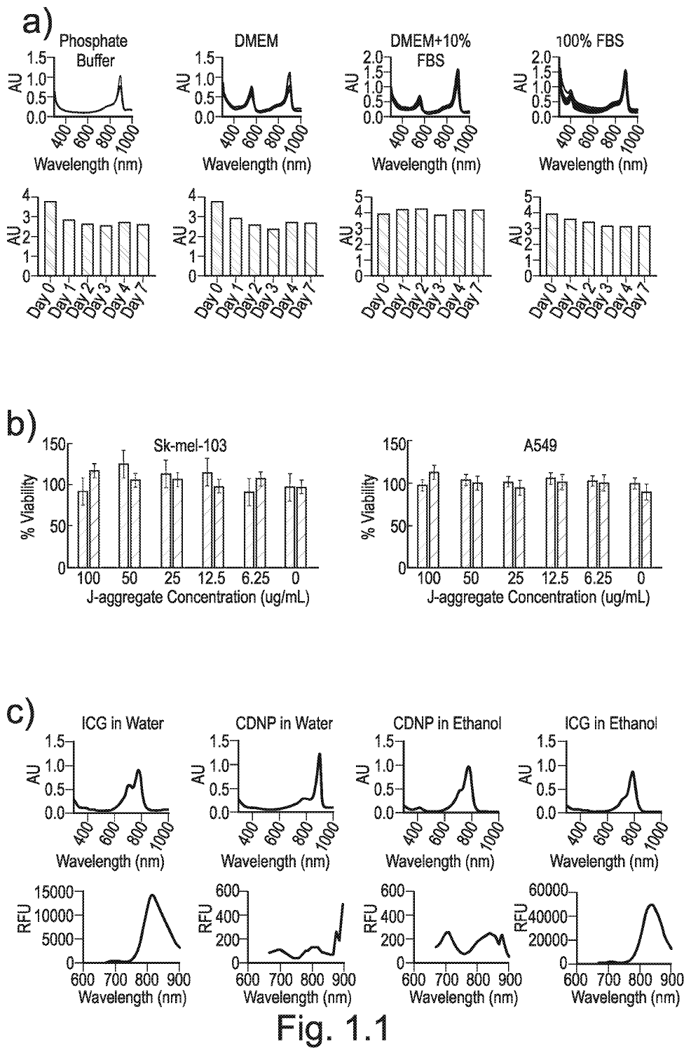

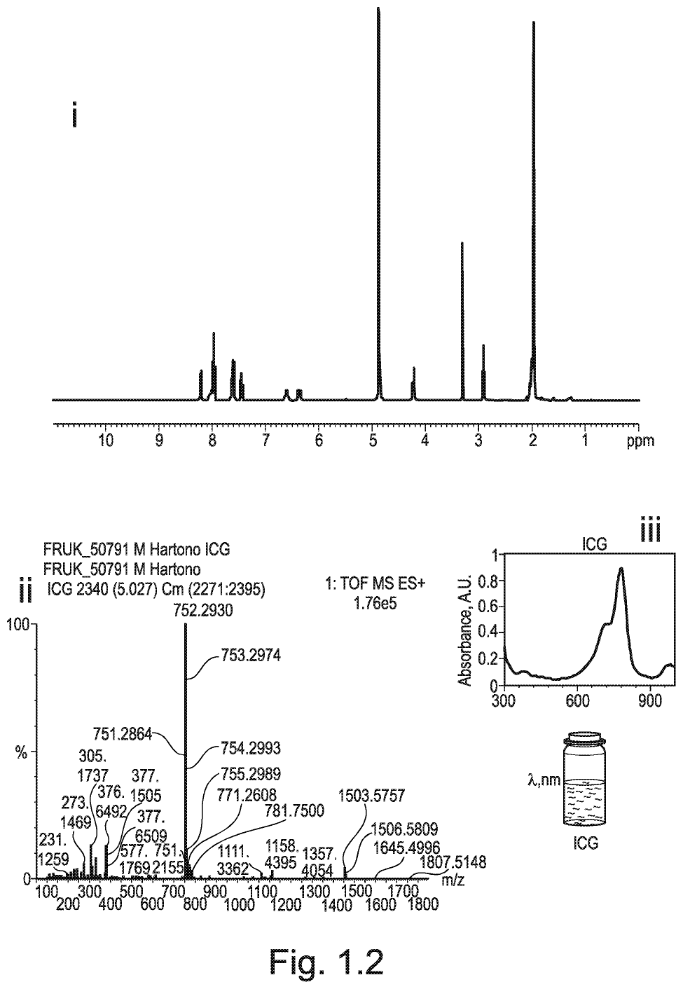

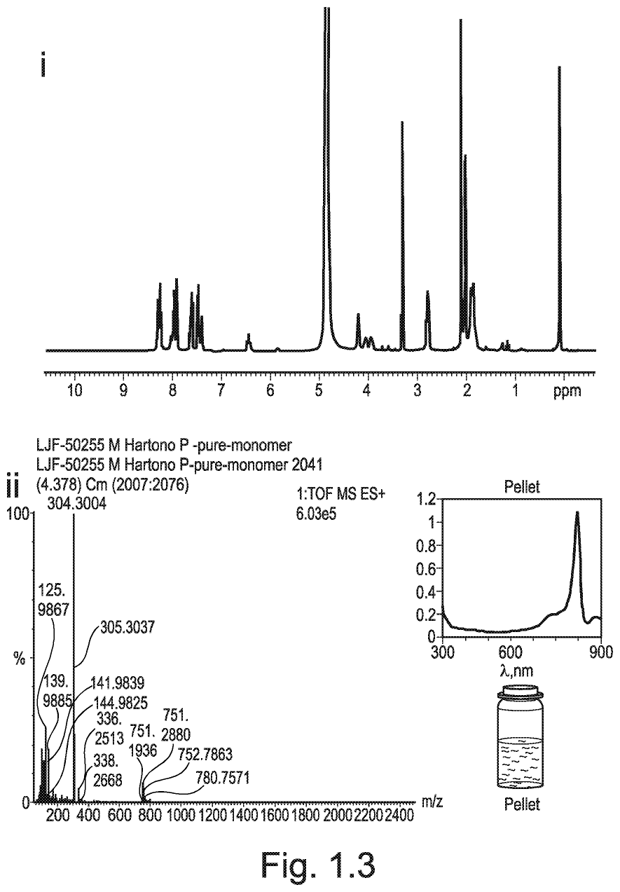

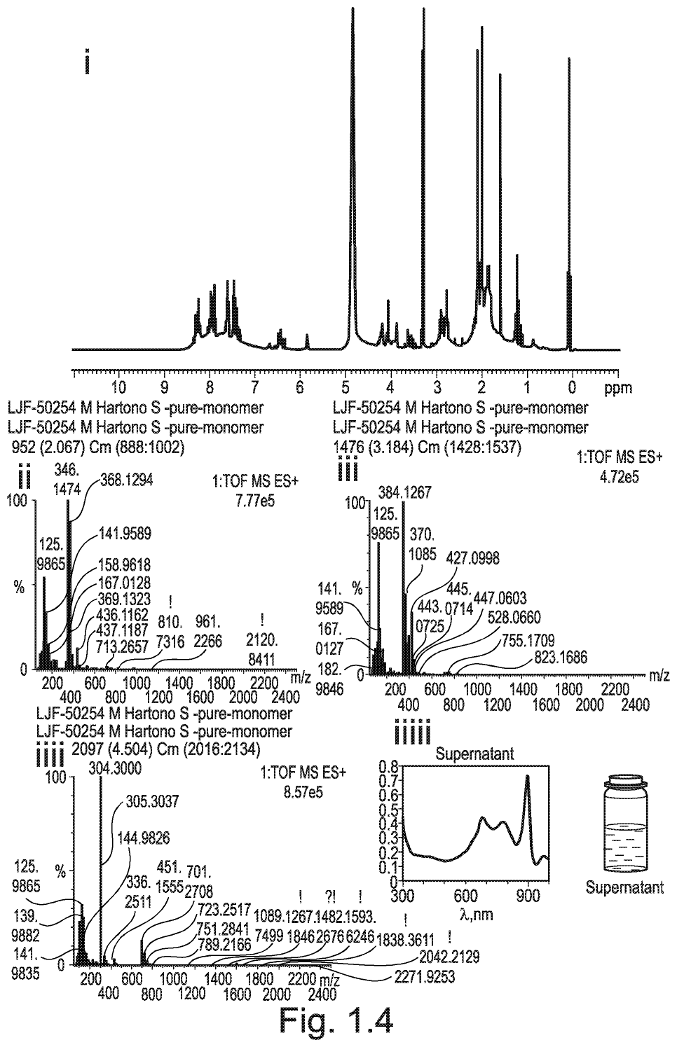

Cryo TEM shows particles with a size of 69 nm+16 nm (FIG. 1c) with absorbance maximum at 895 nm (FIG. 1b). A fluorescent emission peak is also obtained with maximum at 710 nm compared to the monomer of ICG at 820 nm. Photoacoustic spectra of cyanine dye nanoparticles in a phantom were obtained to explore the ability of cyanine dye nanoparticles for in vivo imaging and in particular use of photoacoustic imaging. The results showed 4-fold enhancement of the signal in comparison with ICG monomer as well as red shift of max absorbance (FIG. 1e).

The cyanine dye nanoparticles were stable in water and biological buffers (DMEM, FBS, and PBS) but can be broken apart by addition of a non-polar solvent with UV-VIS resembling the monomer spectrum. However, the presence of two fluorescent peaks indicates that cyanine dye nanoparticles are not completely degraded. This was further investigated by NMR and LC/MS indicating that nanoparticles fall apart into ICG monomers with a characteristic peak of 752+1m/z. NMR and mass spectrum show that the parent mass matches that of ICG at 751+1m/z. However, the NMR spectrum is the same as the dimer structure indicating a smaller aggregate or dimer structure. Unlike the pellet that contains pure cyanine dye nanoparticles, the supernatant obtained after centrifugation had significantly different UV-Vis spectrum and the MS and NMR analysis showing the presence of numerous chemical species.

Prior to in vitro cell studies, stability and toxicity of cyanine dye nanoparticles were evaluated indicating stability in aqueous solution of deionized water over a period of months. In addition, they remain stable for over 96 h at 37° C. in biological media such as DMEM and FBS (FIG. 1.1a). Cell toxicity studies using A549 and SK-MEL-103 cell lines showed no significant impact on cell viability and in a concentration range from 6.25 μg/mL to 100 μg/mL (FIG. 1.1b).

### Photoacoustic Properties of Cyanine Dye Nanoparticles

Photoacoustic measurements were performed using a commercial photoacoustic tomography (PAT) system (inVision256-TF; iThera Medical GmbH) and tissue mimicking phantoms that closely mimic the optical and acoustic properties of biological tissues. Photoacoustic signals of the ICG monomer and cyanine dye nanoparticle were compared.

### Cell Lines

The A549 (human lung adenocarcinoma) cell line was obtained from the European Collection of Authenticated Cell Cultures (ECACC). The SK-MEL-103 (human melanoma) cancer cell line was acquired from the American Type Culture Collection (ATCC). These cell lines were maintained in DMEM (Sigma) and supplemented with 10% fetal bovine serum (FBS). For senescence induction, SK-MEL-103 cells were supplemented with the same media containing palbociclib (PD033299, Pfizer Inc.) at 5 μM for 7 days. A549 cells were supplemented with the same media containing 15 μM cisplatin (Stratech) for 10 days or 10 uM palbociclib (PD033299, Pfizer Inc.) for 10 days. ER: MEK IMR90 (ITM) cells were cultured in phenol red-free DMEM (Sigma) supplemented with 10% FBS, 2 mM I-Glutamine and 1 mM sodium pyruvate (Sigma). This cell line was induced to become senescent with the addition of 200 nM 4-hydroxytamoxifen for 72 hours. Wi-38 cells were purchased from American Type Culture Collection (ATCC). This cell line was maintained in MEM (Thermo Fisher) supplemented with 10% FBS, 2 mM I-Glutamine and 1 mM sodium pyruvate (Sigma). All cell lines were incubated in 20% O2 and 5% CO₂ at 37° C. Cells were routinely tested for mycoplasma using the universal Mycoplasma Detection Kit (ATCC) or by RNA-capture ELISA. For experiments with cells, cisplatin (Stratech) was reconstituted in sterile phosphate-buffered saline (PBS); palbociclib was reconstituted in DMSO.

### Flow Cytometry

LSR Fortessa (BD-Becton Dickinson) was used for flow cytometry analysis. For cyanine dye nanoparticle and small molecule studies, 6 well plates were used and seeded 200,000 cells per well. Once cells were attached, culture medium was changed to DMEM supplemented with 0.2% FBS and cells were exposed cyanine dye nanoparticles for 12-16 hours. After this, cells were trypsinised and re-suspended in PBS buffer with 2% FBS. At least 10,000 live events were collected for each condition. DAPI (Sigma) was added to exclude dead cells. The analysis of all flow cytometry data was performed using FlowJo v10 (Treestar, OR). Unstained control and senescent cells were used as reference for each independent experiment. For ICG the 640 nm laser was used with an emission window of 750-810 nm. For the cyanine dye nanoparticle, the 640 nm laser was used with an emission window of 708-753 nm.

### Confocal Microscopy

Images were acquired on a Leica SP5 confocal microscope using a 20×HCX PL APO 0.5 NA dry objective or a 40×HCX PLAPO 1.3 NA oil immersion objective. Lysotracker and Mitotracker was detected by using excitation wavelength of 488 nm (Argon laser) and with a detection window between 510-530 nm. Cyanine dye nanoparticles were detected by using excitation wavelength of 633 nm (argon laser) and with a detection window between 680-720 nm. Cells without dyes and cyanine dye nanoparticles were used in parallel as autofluorescence controls using the corresponding excitation and detection wavelengths. Images were analysed with LAS AF Lite. For ex vivo, mice were sacrificed 6 h post-injection and tumours placed in a cryomold containing OCT (Leica Biosystems) and frozen in dry ice for 30 min. Frozen OCT sections were washed in PBS and then imaged directly using the above method. The argon laser was used to evaluate autofluorescence of the tissue. Untreated tumours, as well as treated tumours but without cyanine dye nanoparticle injection were used as controls.

### Organelle Co-Localisation Analysis by Confocal Microscopy

Cells were trypsinised and re-plated in flat-bottom μ-clear 96-well plates (Greiner Bio-One, #655087). Cells were seeded at a density of 5,000-6,000 control and 4,000-6,000 senescent cells per well. Once cells were attached, culture medium was changed to DMEM supplemented with 0.2% FBS and exposed cyanine dye nanoparticles for 12-16 hours. In a range from of 100 to 10 μg/mL. After, cells were washed 3× with PBS for 5 min. Specific organelle stains lyso green DND-26 (Thermo Fisher) and MitoTracker Green FM (Cell Signaling Technology), were used according to their manuals. For nuclei staining, Hoescht at 0.1 μg/mL in PBS was added in prior to analysis.

### Mouse Experiments

All mice were treated in strict accordance with the local ethical committee (University of Cambridge License Review Committee) and the UK Home Office guidelines. Tumour xenografts were established using SK-MEL-103. Cells were trypsinised, counted with a haemocytometer, and injected subcutaneously (10⁶ cells in a volume of 100 μL per dorsolateral flank) in 8- to 10-week-old athymic nude female mice (Crl:NU(NCr)-Foxn1^{nu}) purchased from Charles River Laboratories. Tumour volume was measured every 2 days with a caliper and calculated as V=(a×b 2)/2 where a is the longer and b is the shorter of two perpendicular diameters. Palbociclib (Pfizer Inc.) was dissolved in 50 mM sodium lactate at 12.5 mg/mL and administered by daily oral gavage at the indicated doses. 200 uL of cyanine dye nanoparticles (1 mg/mL) and 200 μL of ICG (1 mg/mL) were injected by the tail vein in DMEM (Thermofisher, no phenol red).

### Ex Vivo Imaging by IVIS

An IVIS Spectrum Imaging System (Perkin Elmer Inc) was used for ex vivo fluorescence imaging. For ex vivo imaging, mice were sacrificed by cervical dislocation and organs and tumour xenografts were analysed immediately after harvesting. ICG was detected using an excitation wavelength of 710-740 nm and emission bandpass from 810-830 nm. Cyanine dye nanoparticles were detected using an excitation wavelength of 605 nm and emission of bandpass of 700-720 nm. Fluorescence imaging quantification was performed by Living Image 3.2 software (Perkin Elmer Inc). A region of interest area (ROI) was drawn over the fluorescent signal in tumours. Fluorescence activity is measured in photons per second per square centimetre per steradian (p/s/cm²/sr).

### Immunoblotting

Cell lysis was performed using RIPA buffer (Sigma) supplemented with phosphatase inhibitors (PhosSTOP™ EASYpak Phosphatase Inhibitors Cocktail, Roche) and protease inhibitors (complete™ Protease Inhibitor Cocktail, Roche). Proteins were quantified and separated by SDS-PAGE and transferred to polyvinylidene difluoride (PVDF) membranes (Millipore) according to standard protocols. Membranes were immunoblotted with antibodies against p21 and p53 from Santa Cruz Biotechnology, and phospho-Rb (pRBS780) from Cell Signaling. After incubation with the primary antibody overnight, membranes were washed and incubated with secondary HRP-conjugated AffiniPure antibodies (Jackson ImmunoResearch) for 1 hr at room temperature and subsequently incubated with Enhanced Chemiluminescence Detection solution (Amersham).

### Senescence Associated β-Galactosidase (SA-β-Gal)

SA-β-Gal staining was performed using the Senescence β-Galactosidase Staining kit (Cell Signaling), following the manufacturer instructions. Briefly, cells were fixed at RT for 15 min with a 2% formaldehyde, washed with PBS and incubated overnight at 37° C. with the staining solution containing X-gal in N—N-dimethylformamide (pH 6.0). Pictures were taken using a Wide Field Zeiss Axio Observer 7. For Tissue Cryosections this method was repeated however, the pH was modified to 4.0 and incubated only for 4 hours.

### Results and Discussion

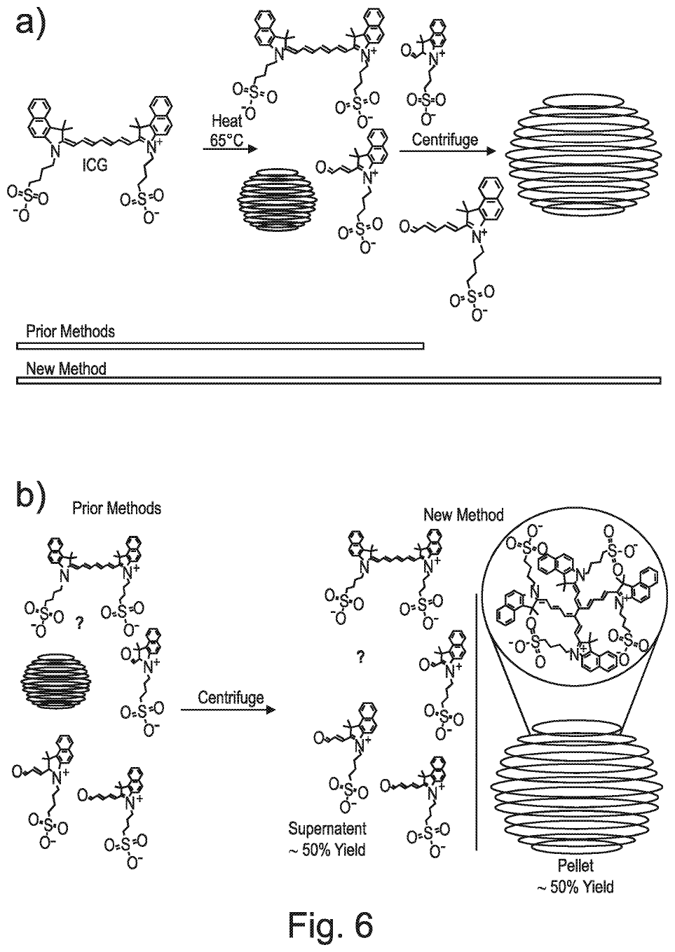

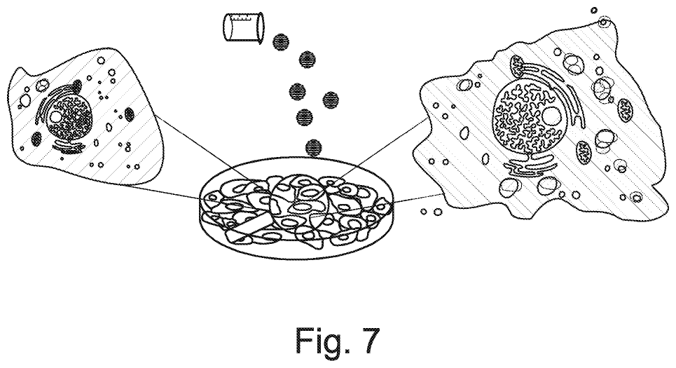

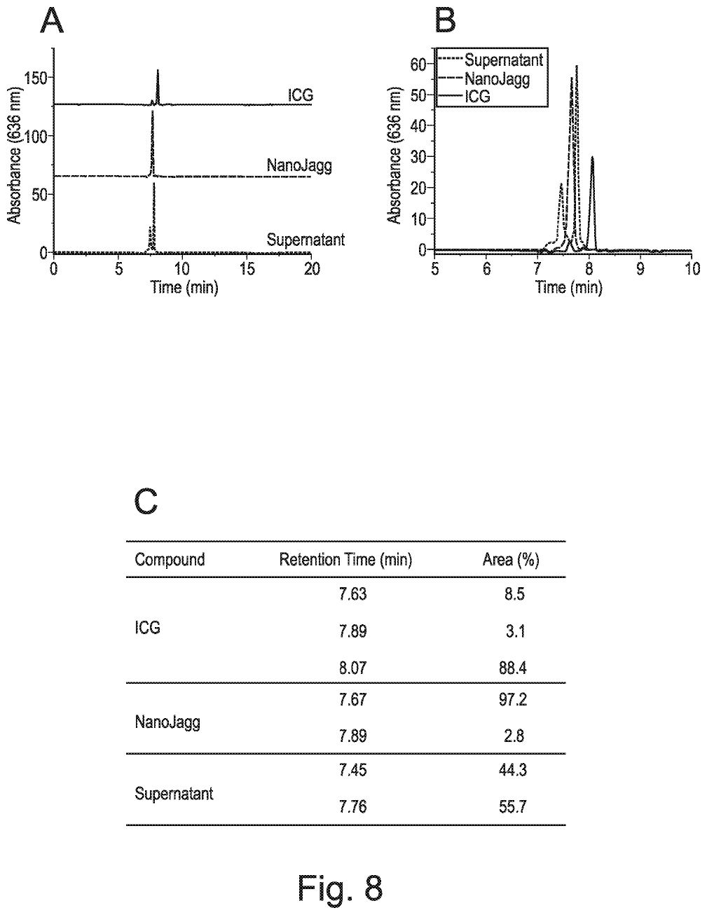

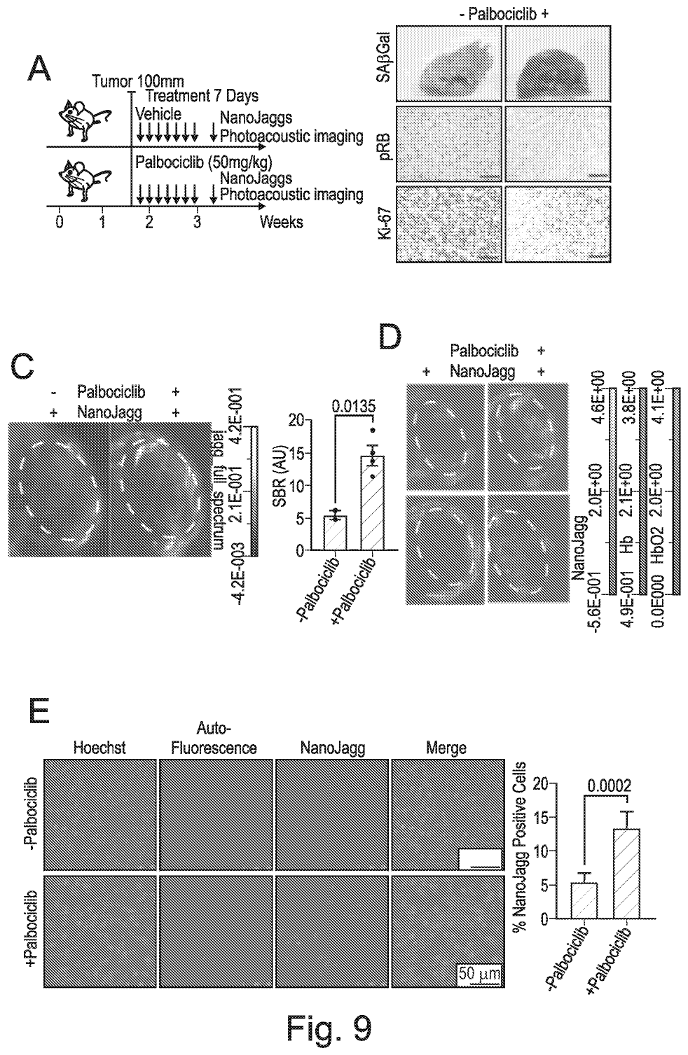

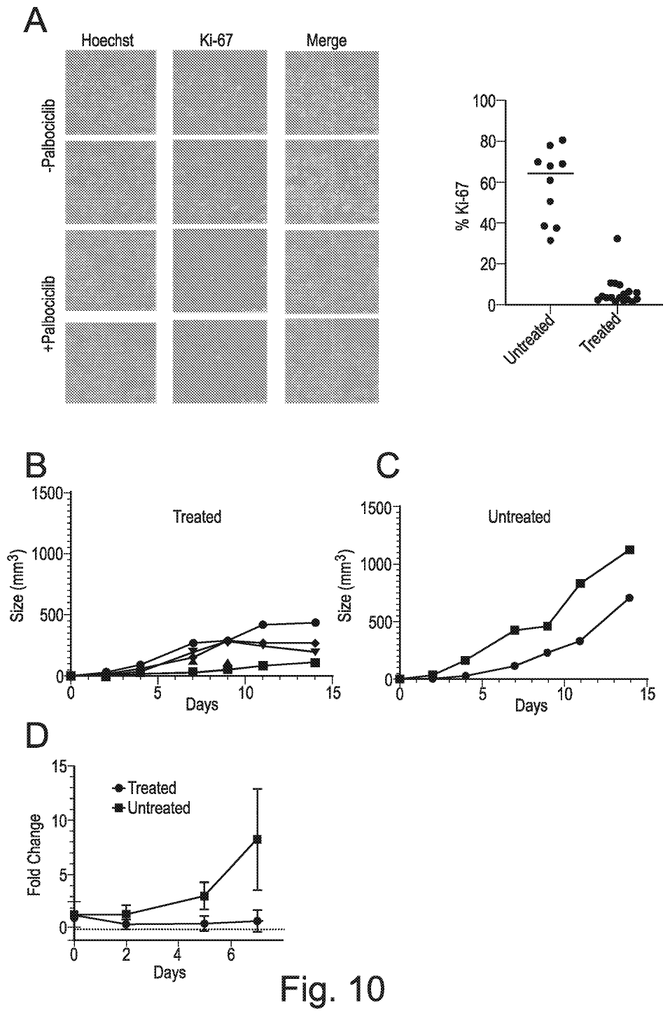

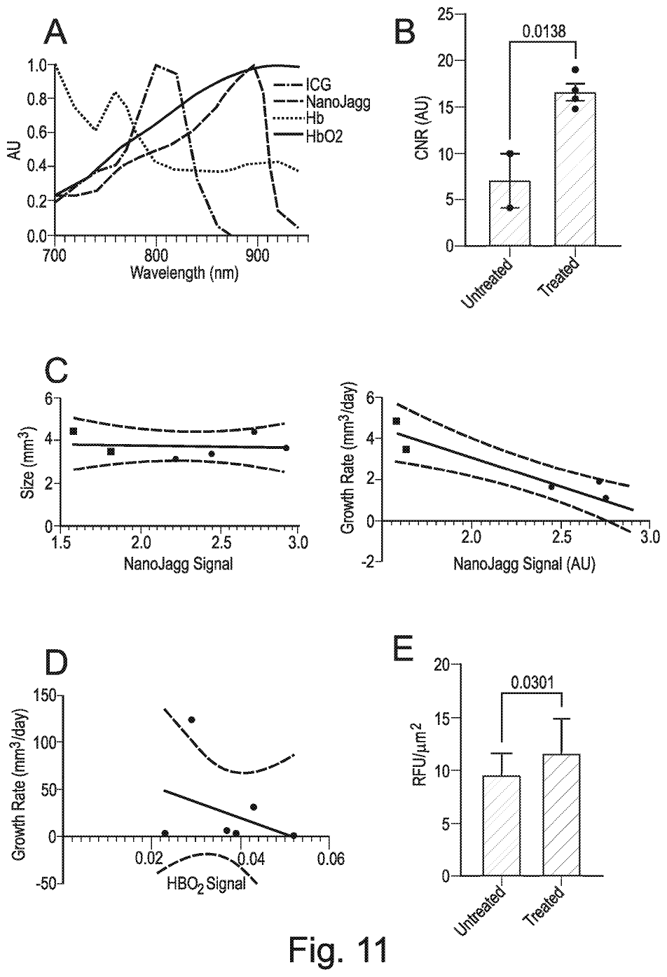

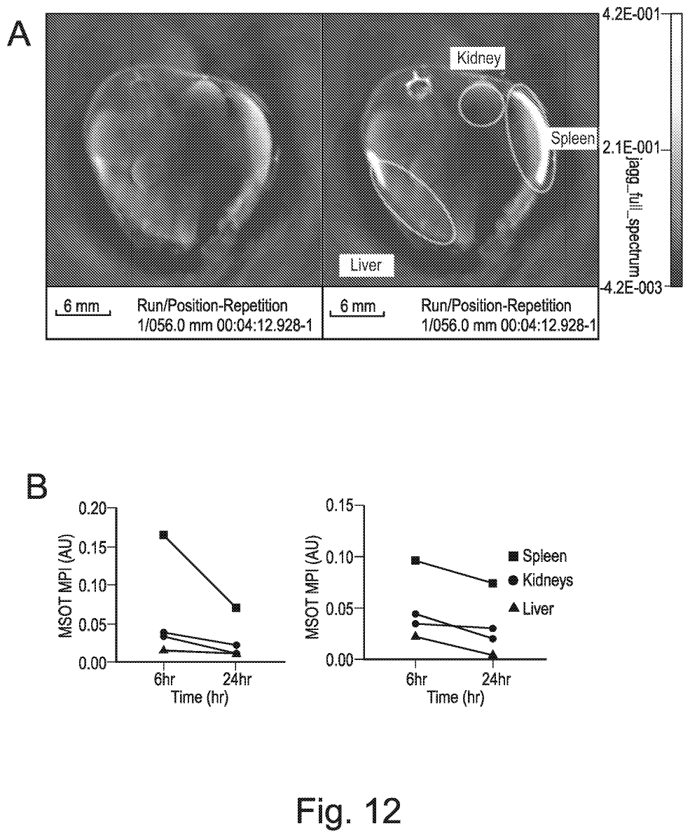

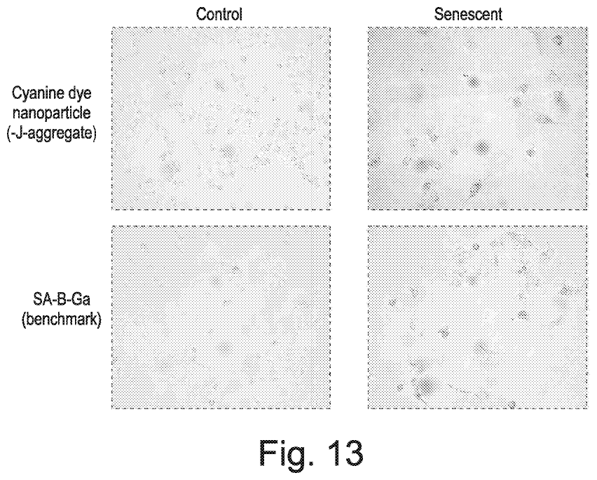

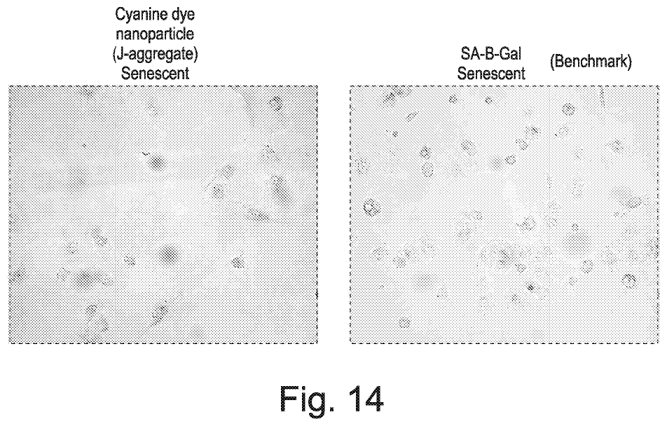

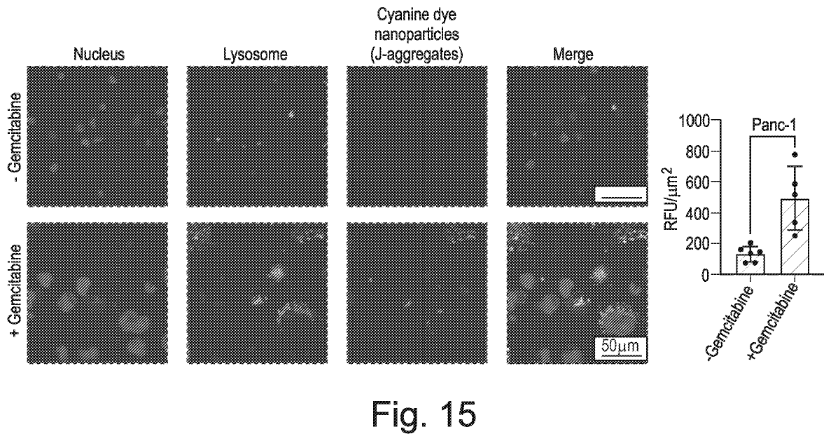

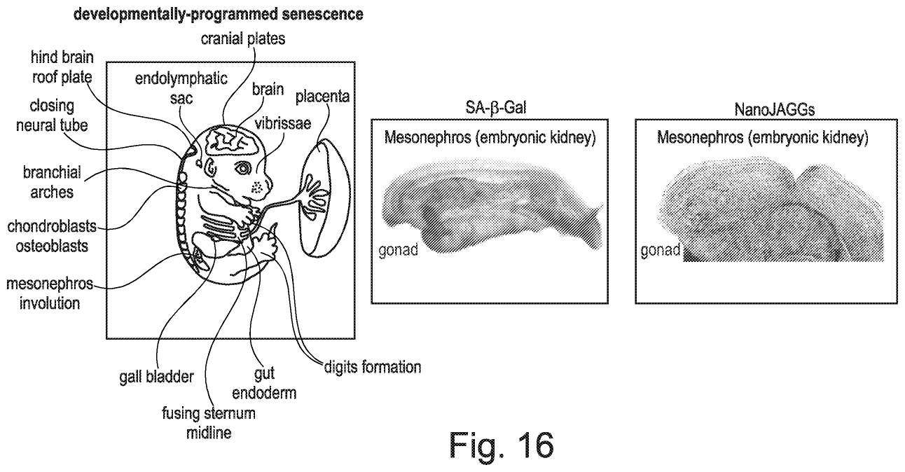

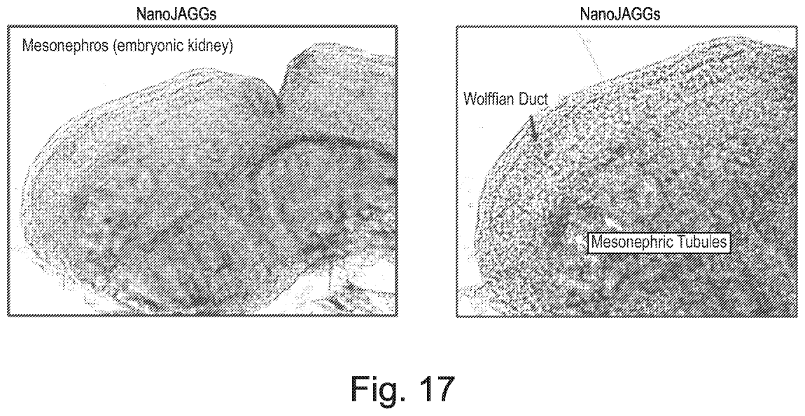

When compared with previous methods, the present invention introduces another step of purification in the form of centrifugation and washing. This is depicted in FIG. 6. Previous methods such as Liu et al (2017), use the method first created by Rotermund et al (1997), wherein ICG is dissolved at a concentration of 1.5 mM and placed in a ultrasonic bath for 10 min. The solution is then heated to 65-90° C. for 32 hrs and cooled to 20° C. for 6 days. This resulting solution is considered to be a cyanine dye nanoparticle aggregate because of the characteristic J-band at 895 nm. Other methods add a step here using dialysis, or filtration. However, none of these methods employ NMR or mass spectrometry to further characterise the solutions. The steps of the present invention comprise purification in the form of centrifugation and washing of the resulting pellet three times at 18,000 rpm (4° C.) for 30 min. Analysis showed that these are the cyanine dye nanoparticles, and other methods likely have a complicated mixture of Modified ICG, dimers, trimers and cyanine dye nanoparticles.

In the methods of the present invention, the composition is centrifuged, and the cyanine dye nanoparticles are concentrated in the pellet, while the supernatant is left containing monomers and likely degradation products. This is evident from observing the absorbances and optical properties of ICG, the supernatant and the pellet in FIGS. 1.2, 1.3 and 1.4.

From FIGS. 1.2, 1.3 and 1.4, it is clear that the pellet contains cyanine dye nanoparticles while the supernatant, contains monomers or not fully formed nanoparticle aggregates. This difference was characterised chemically. The NMR and mass spectrum of ICG are shown in FIG. 1.2. After the reaction and followed by dialysis and centrifugation the supernatant NMR spectrum changes from ICG. Although the dimer mass of 785 or 1501 m/z was not found, other masses were identified indicating that ICG in some cases is degrading into smaller products. However, based on its absorption spectra these smaller products are also aggregating. As the supernatant makes up 50% of yield it is likely to make a substantial contribution to any biological application. The cyanine dye nanoparticle pellet's NMR (FIG. 1.3) is different to the NMR of pure ICG (FIG. 1.2) and the supernatant (FIG. 1.4), with its spectrum matching the reported spectra in Mindt et al. (2018) and Rüttger et al (2019), who identify the ICG dimer using mass spectroscopy and NMR. However, its mass spectra contain only that of ICG. Further this has been confirmed in a separate study by another mass spec core, where both the ICG and cyanine dye nanoparticle have similar mass spectra, and fragmentation patterns. Thus, the methods of the present invention result in chemically pure cyanine dye nanoparticles from prior methods.

### Characterization of Senescent Cells

Senescent cells were characterized using three approaches:
(a) monitoring the enhancement of SAβGal activity;
(b) lack of proliferation using growth curves; and
(c) protein expression.

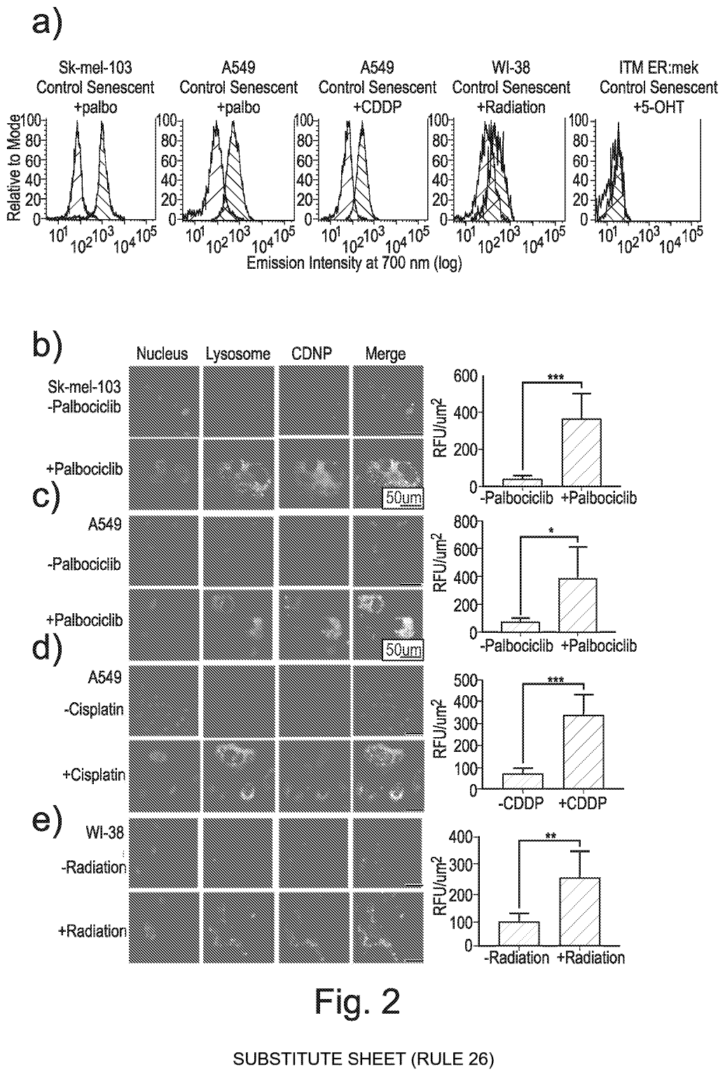

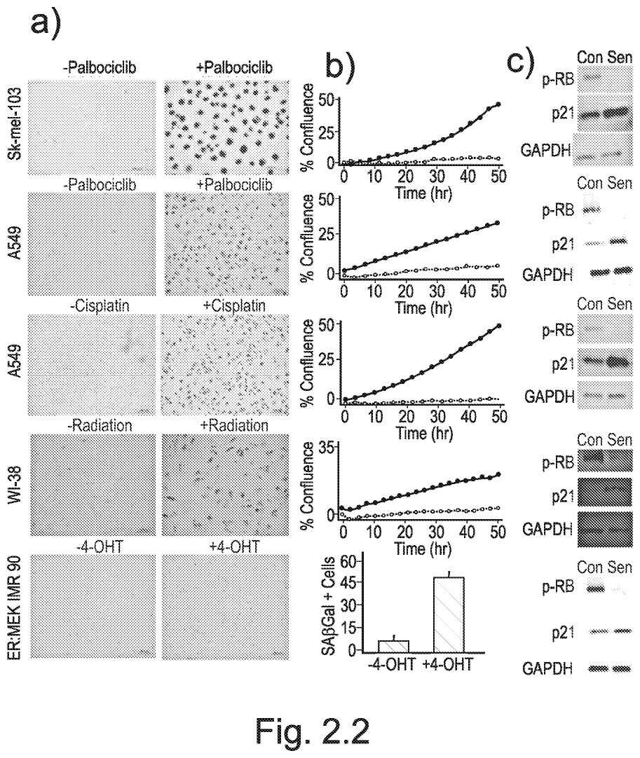

The absence of the phosphorylated retinoblastoma protein (p-RB) was used as well as the presence of p21 and p53 proteins. In all of the models there was an increased SA-β-Gal activity (FIG. 2.2a). Furthermore, in all cases there was a reduction in the proliferative ability of senescent cells monitored over 48 hours (FIG. 2.2b). Moreover, in all cases a decrease in expression of p-RB, and an increased expression of p21 and p53 is observed (FIG. 2.2c).

### Validation of Cyanine Dye Nanoparticle Detection of Senescent Cells In Vitro

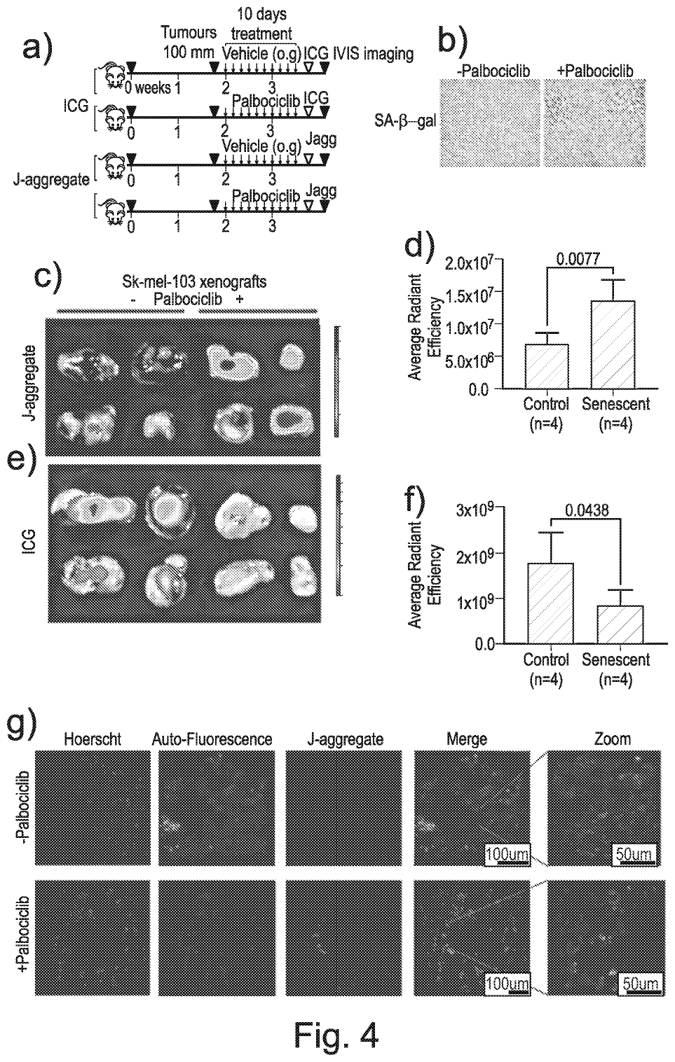

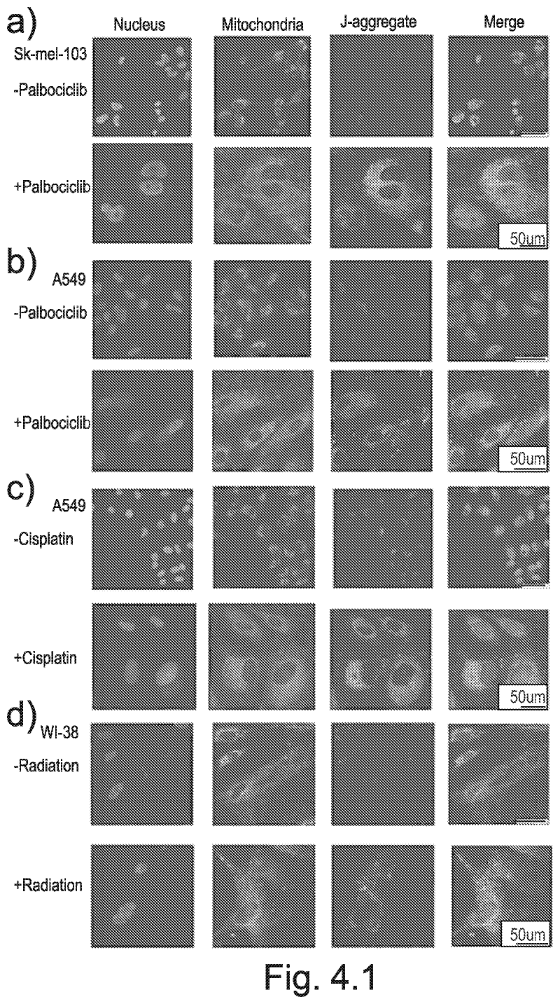

In the in vitro cultures, cyanine dye nanoparticles accumulate in models of chemotherapy and radiation induced senescence, with a larger accumulation in chemotherapy induced senescent cancer cells (FIG. 2a). This data is repeated using confocal microscopy, and all quantifications are controlled by cell size. In all models, senescent cells significantly accumulate more cyanine dye nanoparticles even after controlling the size (FIG. 2b-e). Furthermore, in all models the cyanine dye nanoparticles colocalize in the lysosomes. The cyanine dye nanoparticles did not colocalize with the mitochondria in any of the cell models (FIG. 4.1a-d), demonstrating that the cyanine dye nanoparticles are able to successfully target senescent cells.

### Small Molecule Accumulation in Senescent Cells In Vitro

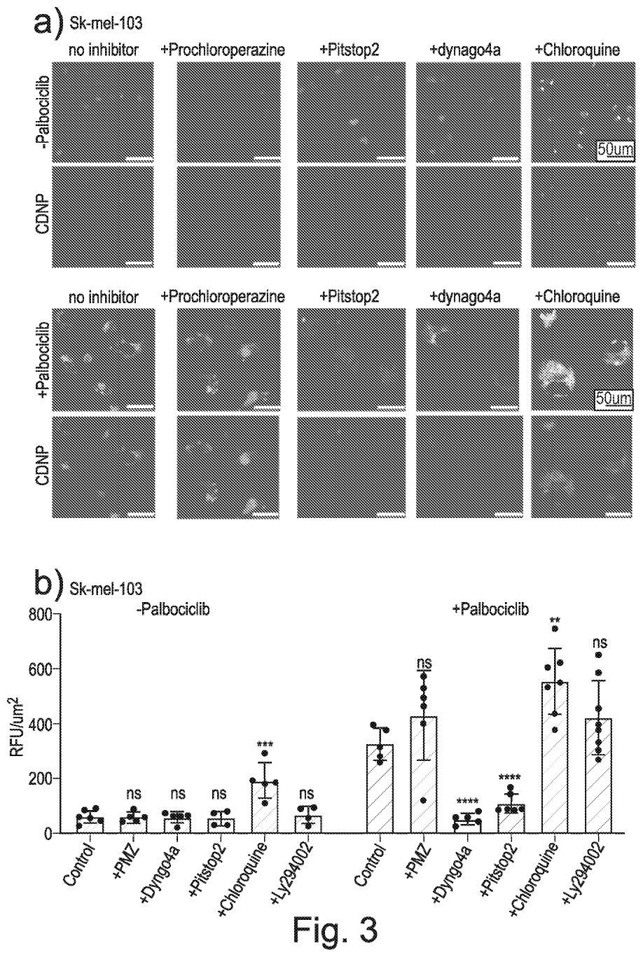

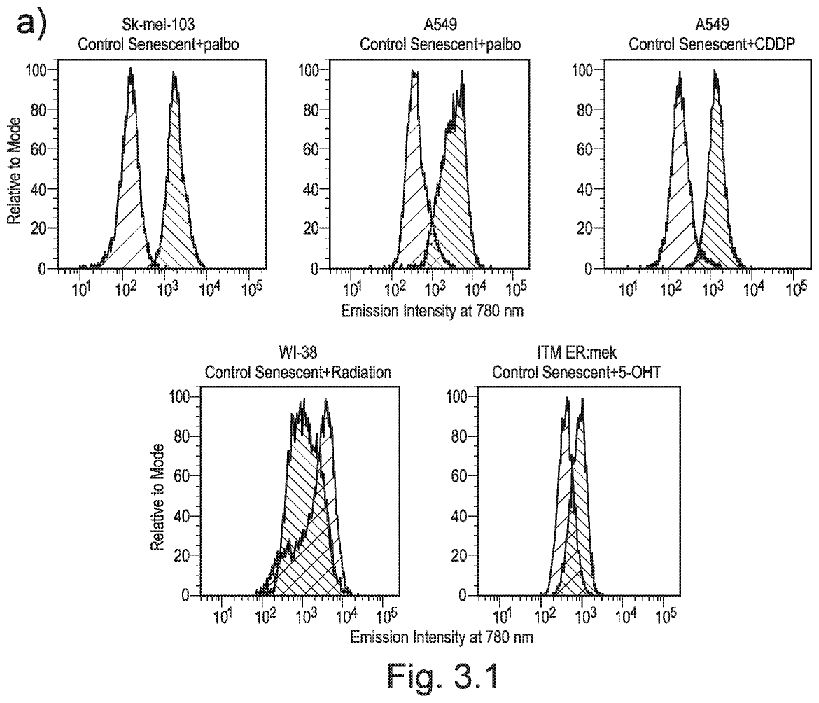

As a comparison to the experiments with cyanine dye nanoparticles, ICG was also used. In all models, senescent cells had accumulation of ICG (FIG. 3.1a).

### Endocytosis of Cyanine Dye Nanoparticles

Cyanine dye nanoparticles were incubated with cells and in the presence of different inhibitors. Cyanine dye nanoparticle entry was not inhibited with the addition of prochloroperazine, a clatherin dependent endocytosis inhibitor. Pitstop 2, on the other hand, was a significant inhibitor of endocytosis. Similarly, dynago4a, an inhibitor for dynamin, significantly inhibited the uptake of cyanine dye nanoparticles.

On the other hand, chloroquine significantly enhances the uptake of cyanine dye nanoparticles, and/or reduces the breakdown, as it is an autophagy inhibitor and expands the lysosomal compartment. This is the case for the control cells as well.

The role of micropinocytosis in the uptake of these nanoparticles was considered, as many RAS activated cancers show a reliance on this pathway. However, the uptake was not significantly reduced in the presence of a chemical inhibitor, although the overall distribution was changed. It therefore appears that the cyanine dye nanoparticles participate in an active process of cellular uptake likely involving dynamin and clathrin.

### Cyanine Dye Nanoparticle Targeting in Xenografts Fluorescence

To evaluate the targeting ability of the cyanine dye nanoparticles in vivo, tumour xenografts treated with a senescence inducing chemotherapy were used. The experimental set up is shown in FIG. 4a.

Subcutaneous xenografts were created with SK-MEL-103 melanoma cells. Upon tumour formation, mice were treated with Palbociclib for 7 days. This resulted in a high number of senescent cells in the tumor, as measured by the SA-β-Gal activity and absence of the proliferative marker Ki67 (FIG. 4b). The mice were treated in three ways, injection of ICG, injection of cyanine dye nanoparticles or no contrast agent added at day 7. All tumors were then imaged using an IVIS system 6 h after the final injection.

Cyanine dye nanoparticles demonstrate a significantly higher in uptake in senescent tumors than controls (p=0.0077) (FIG. 4c). Furthermore, because of the significantly red emission, it is unlikely that autofluorescence is a confounding factor. The same image in the ICG channel was also measured and did not reveal a significant difference, indicating the ICG is still bound in cyanine dye nanoparticle aggregate structure. ICG on the other hand shows a significantly higher signal in the control tumours (p=0.0438), with growing and non-senescent cells (FIGS. 4c and d).

The accumulation of cyanine dye nanoparticles in senescent tumours was further supported using confocal imaging of the tumour sections (FIG. 4g). These sections show an increased accumulation of the cyanine dye nanoparticle signal localized near the cell nuclei.

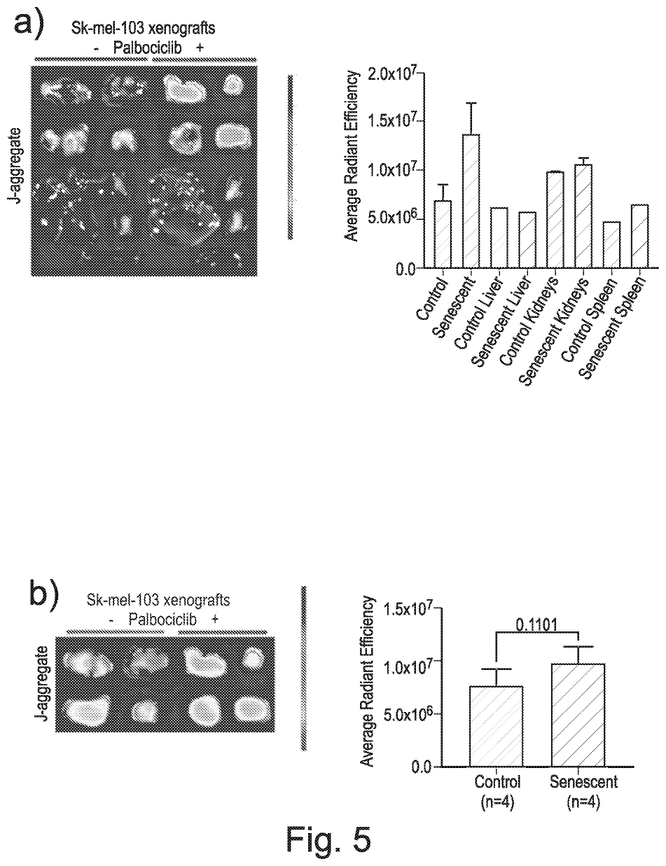

Furthermore, these images show that the signal is different than autofluorescence. Cyanine dye nanoparticles seem to be eliminated via the kidneys as shown in FIG. 5a. These results in vivo demonstrate that cyanine dye nanoparticles target specifically senescent tumours and can be used as a tool to detect senescent lesions in vivo.

### Examples—Part B

### Figures

Throughout the entirety of this specification and accompanying figures, the terms “J-aggregates” and “NanoJaggs” are used interchangeably and will be understood to refer to the cyanine dye nanoparticles of the present invention.

FIG. 8 shows that the cyanine dye nanoparticles (NanoJaggs) are 97% pure by HPLC characterisation. (A) Single component trace of ICG (8.07 min), NanoJaggs (7.67 min), and Supernatent ((7.45 and 7.76 min). (B) Combined HPLC trace to demonstrate difference in retention times. (C) Table of retention times of each pure sample calculated from the area under the curve of each individual trace.

FIG. 9 NanoJaggs act as contrast agents for in vivo photoacoustic imaging of cellular senescence. (A) Experimental design using two groups of mice treated and un-treated with Palbociclib. Both groups were given NanoJaggs after their treatment regime. (B) Tumors stained with X-gal show strong β-galactosidase activity in Palbociclib treated tumors. IHC images of Palbociclib treated tumor slices show a decrease in nuclear pRB and Ki-67 compared to control tumors. Scale bars 100 mm. (C) Representative axial images of control (n=2) and senescent tumors (n=4) treated with NanoJaggs. Quantification shown to the right. Photoacoustic signal is in arbitrary units. SBR is signal/background. A significant difference from the untreated vs Palbociclib treated tumors can be noted. (D) MSOT images showing tumors spectrally unmixed for NanoJagg (yellow), Hb (blue) and HbO2 (red). (E) Confocal images and quantification of NanoJagg positive cells comparing senescent and control mice. In both cases multiple tumors were used, as well as imaged in different parts of each slice. Data represent mean±SD, and a Two-tailed t-test was used to calculate the significance (*p<. 05, ** p<01, *** p<. 001).

FIG. 10 Further characterization of tumor senescence. (A) Ki-67 staining with immunofluorescence. Treated tumors were compared to untreated, through multiple slices and areas. Quantification shown to the right (p<0.0001). (B and C) Growth curves of the untreated and treated tumors. Treated tumors show growth arrest while untreated are proliferating. (D) Same data in B and C represented by fold change, to highlight growth arrest in treated tumors compared with no arrest in untreated tumors. Palbociclib treated tumors again show a marked decrease in proliferation rate indicative of senescence induction.

FIG. 11 Evaluation of NanoJaggs for in vivo photoacoustic imaging. (A) Absorption spectra used for spectral unmixing. The provided spectrum of ICG, Hb, and HbO2 were used, and the measure spectrum of the NanoJaggs was used. (B) Contrast to noise ratio (CNR)) was compared for untreated and treated tumors. Treated tumors had a higher CNR, to untreated (p=0.0138), treated tumors also had a higher SNR than untreated (p=0.0140). (C) NanoJagg signal does not correlate with tumor size (R2=0.015, p=0.7939, ns), but does correlate with the tumor growth rate (R2=0.8, p=0.002, **). (D) HBO2 signal does not correlate with tumor growth rate (R2=0.097, p=0.4952, ns) (E) Quantification of fluorescence per cell in untreated and treated tumors, the fluorescence was more in the treated tumors for each cell (p=0.0301). Data represent mean±SD, and a Two tailed t test was used to calculate the significance (*p<. 05, ** p<. 01, *** p<. 001).

FIG. 12 Evaluation of NanoJagg elimination. (A) Elimination of NanoJaggs is seen via the spleen, liver and kidneys. (B) NanoJagg signal observed in organs over time 6 h and 24 h. After 24 h the amount of signal in these organs drops in two mice.

FIG. 13 shows breast cancer senescence using the breast cancer cell line, MDA-MB-231. Brightfield microscopy images of control MDA-MB-231 and senescent cells induced with Palbociclib, NanoJaggs demonstrate visible accumulation in senescent cells. Brightfield microscopy of control MDA-MB-231 control and senescent cells induced with Palbociclib staining with X-Gal. Senescent cells demonstrate increased staining indicating senescence.

FIG. 14 shows breast cancer senescence using the breast cancer cell line, MCF-7. Brightfield microscopy images of MCF-7 senescent cells induced with Palbociclib, NanoJaggs demonstrate visible accumulation in senescent cells. Brightfield microscopy of control MCF-7 senescent cells induced with Palbociclib staining with X-Gal. Senescent cells demonstrate positive staining indicating senescence.

FIG. 15 shows pancreatic cancer senescence using the pancreatic cancer cell line, Panc-1. Confocal microscopy of both control and senescent cells induced with Gemcitabine. Quantification of total NanoJagg signal in cells indicates statistically significant more NanoJagg accumulation.

FIG. 16 shows cellular senescence in embryonic structures. Contains cartoon of anatomy of mouse embryo, image of mesonephros stained with x-gal, and image of mesonephros stained with NanoJaggs

FIG. 17 shows cellular senescence in embryonic structures (mesonephros) as stained by NanoJaggs.

### Materials and Methods

### Cyanine Dye Nanoparticle Synthesis

Aqueous ICG (Acros Organics, 10321541) solution (0.75 mM, 10 mL) was sonicated for 10 min then heated to 65° C. under stirring (500 rpm). The reaction was monitored by UV-Vis spectrophotometer (transition of 780 nm to 895 nm) and upon completion (˜24 h) the reaction mixture was centrifuged and washed three times at 17000 rpm (31000×g) at 4° C. for 30 min in a Sorvall LYNX 4000 high speed centrifuge. The obtained pellet of this reaction was redispersed in deionized water and filtered through a 0.2 μm filter and lyophilized to obtain dark green cyanine dye nanoparticles (5.4 mg, 54% yield). Lyophilization was carried out using a Telstar LyoQuest benchtop freeze dryer (0.008 mBar, −70° C.). The hydrodynamic size and zeta potential of the cyanine dye nanoparticles were measured using a Zetasizer Nano Range instrument (Malvern Panalytical). To determine the chemical composition, LC-MS and ¹H NMR were conducted on the obtained cyanine dye nanoparticles and the supernatant obtained during the centrifugation. Using HPLC to assess the dimer content, the purity of NanoJaggs was determined at 97%, in contrast again the supernatant is seen to contain multiple peaks at different retention times (FIG. 8).

ICG: ¹H NMR (400 MHZ, MeOD): δ 8.23 (d, J=8.6 Hz, 1H), 8.13-7.94 (m, 2H), 7.70-7.56 (m, 1H), 7.54-7.41 (m, 1H), 6.73-6.49 (m, 1H), 6.39 (d, J=13.4 Hz, 1H), 4.25 (t, J=6.5 Hz, 1H), 2.94 (t, J=6.8 Hz, 1H), 2.25-1.74 (m, 6H). HRMS: calculated for C₄₃H₄₇N₂O₆S₂⁻ (M+H)⁺: Mass predicted: 752.29; Found: 752.2930

Cyanine dye nanoparticles (pellet): ¹H NMR (400 MHZ, MeOD) δ 8.37-8.25 (m, J=15.7, 8.0 Hz, 4H), 8.10-7.92 (m, 6H), 7.67 (dd, J=18.5, 8.4 Hz, 4H), 7.56-7.43 (m, 4H), 6.53-6.41 (m, 2H), 5.91 (d, J=13.9 Hz, 1H), 4.30-4.21 (m, 2H), 4.14-4.06 (m, J=7.4 Hz, 2H), 4.02-3.92 (m, 2H), 2.91-2.75 (m, J=14.8, 8.2 Hz, 5H), 2.14 (s, 5H), 2.07 (d, J=8.2 Hz, 6H), 2.00-1.79 (m, 9H). HRMS: calculated for C₈₆H₉₄N₄O₁₂S₄₂⁻ (M+H)⁺: Mass predicted: 751.28; Found: 751.2880

Supernatant obtained during cyanine dye nanoparticle purification: ¹H NMR (400 MHz, MeOD) δ 8.38-8.20 (m, 1H), 8.19-8.11 (m, 1H), 8.08-7.87 (m, 2H), 7.84-7.76 (m, 1H), 7.74-7.57 (m, 1H), 7.56-7.39 (m, 1H), 7.40-7.32 (m, 1H), 6.52-6.33 (m, 1H), 3.94-3.85 (m, 1H), 3.03-2.70 (m, J=31.1, 24.2, 17.8, 10.9 Hz, 2H), 2.31-1.71 (m, 6H), 1.68-1.55 (m, J=7.7 Hz, 1H), 1.38-1.08 (m, 1H).

### Spectrophotometry

UV-Vis absorption spectra were obtained with an Agilent Cary 300 Spectrophotometer, Spark and Infinite 200 Pro (Tecan) plate reader. Fluorescence emission spectra were obtained using a Varian Cary Eclipse Fluorescence Spectrophotometer as well as The Spark (TECAN) and Infinite 200 Pro (Tecan).

### SEM, TEM and Cryo-TEM

SEM and TEM images were obtained using a FEI Verios 460. Samples were suspended in water and drop cast (2 μL) on lacey carbon copper grids (Agar Scientific). Cryo-TEM micrographs were obtained using a Thermo Scientific (FEI Company) Talos F200X G2 microscope operated at 200 kV. Images were recorded on a Ceta 4k×4k CMOS camera and processed with Velox software. Specimens for investigation were prepared through vitrification by plunge freezing of the aqueous suspensions on copper grids (300 mesh) with lacey carbon film. Prior to use, the grids were glow discharged using a Quorum Technologies GloQube instrument at a current of 25 mA for 60 s. Suspensions of the samples (2.5 μL of a 1 mg/mL solution) were pipetted onto the grid, blotted using filter paper and immediately frozen by plunging in liquid ethane utilizing a fully automated and environmentally controlled blotting device, Vitrobot Mark IV. The Vitrobot chamber was set to 4° C. and 95% humidity. Samples after vitrification were kept under liquid nitrogen until they were inserted into a Gatan Elsa cryo holder and analyzed in the TEM at −178° C.

### Mass Spectrometry

LC-MS was performed using a Waters' Xevo G2-S bench top QTOF and was performed by the Department of Chemistry Mass Spectrometry Service. Samples were first dissolved in HPLC-grade methanol at a concentration of 10 μg/mL to ensure the aggregate structure was dissolved.

### ¹H NMR

¹H NMR measurements were carried out using 400 MHZ QNP Cryoprobe Spectrometer (Bruker) by the NMR service of the Department of Chemistry, University of Cambridge. Samples were dissolved in deuterated methanol to ensure the aggregate structure was dissolved.

### Colloidal Stability of Cyanine Dye Nanoparticles

Cyanine dye nanoparticles were dissolved in deionized water at a concentration of 1 mg/mL. This was then diluted over a range of concentrations from 100 μg/mL to 1 μg/mL in 1X phosphate buffer (PBS) pH 7.4, 100% FBS, DMEM with 10% FBS and DMEM with 0.2% FBS. Every day for a week the absorbance ratio of 895 nm for the cyanine dye nanoparticle aggregate peak was measured and compared that to 780 nm or ICG max absorption. This was used to calculate how stable these nanoparticles were in solutions.

### Cell Lines

See Examples—part A

### Senescence Induction

For senescence induction, SK-MEL-103 cells were supplemented with the same media containing Palbociclib (PD0332991, MCE) at 5 μM for 7 days. A549 cells were supplemented with the same media containing 15 μM Cisplatin (Stratech) for 10 days or 10 μM Palbociclib (PD0332991, MCE.) for 10 days. WI-38 cells were treated with 10 Gy X-ray irradiation and maintained for 10 days. Cisplatin (Stratech) was reconstituted in sterile PBS and Palbociclib in DMSO.

### Immunoblotting

See Examples—part A

### Senescence-Associated β-Galactosidase (SA-β-Gal)

SA-β-Gal staining was performed using the Senescence β-Galactosidase Staining kit (Cell Signaling), following the manufacturer instructions. Briefly, cells were fixed at RT for 15 min with 2% formaldehyde, washed with PBS and incubated overnight at 37° C. with the staining solution containing X-gal in N,N-dimethylformamide (pH 6.0). The next day cells were washed 3× with PBS for 2 minutes, and finally PBS was added to the cells for imaging. Pictures were taken using a Wide Field Zeiss Axio Observer 7. For tissue cryosections this method was repeated, however the tissue was incubated with X-gal for 4-6 h.

### Brightfield Imaging of Cyanine Dye Nanoparticles

Cells were plated in 6-well plates (Eppendorf) at a concentration of 200,000 cells/well. The cells were incubated overnight in various concentrations of cyanine dye nanoparticle solutions (10 μg/mL-100 μg/mL). Typically, most experiments were conducted at a concentration of 50 μg/mL. After 12 h cells were washed 3× with PBS for two minutes and incubated in phenol red-free DMEM (Gibco, 11594416) for imaging. Imaging was performed using a Wide Field Zeiss Axio Observer 7.

### Cell Viability Assays

Cell viability was determined using CellTiter-Blue assay (Promega). CellTiter Blue uses the reduction of resazurin to resorufin to measure cell viability. Cells were seeded in a 96-well plates (Eppendorf) 3,500 control cells/well and 5,000 senescent cells/well. After 24 h, cyanine dye nanoparticles were added to the cells for 72 h at a range of 1 μg/mL to 100 μg/mL. After 72 h, 4 μL CellTiter-Blue reagent was added to each well. After incubation for 2 h, the absorbance and fluorescence were recorded for each well on the Infinite 200 Pro (Tecan) using 560/590 excitation emission. The viability studies were conducted in 4 technical replicates and 3 biological replicates. The viability was calculated according to the following equation: 100×((Sample−Negative Control)/(Untreated Cells−Negative Control)). This was shown as percent viability.

### Flow Cytometry

See Examples—part A

### Confocal Microscopy

Cells were seeded in a 96 well plates (Eppendorf) 3,500 control cells/well and 5,000 senescent cells/well in 96 well μ-clear plates (Greiner Bio-One). After 24 h cells were incubated with cyanine dye nanoparticles at concentrations of 10-100 μg/mL. Confocal images were acquired on a Leica SP5 confocal microscope using a 20X HCX PL APO 0.5 NA dry objective or a 40X HCX PL APO 1.3 NA oil immersion objective. Hoechst (ThermoFischer) was used to specifically dye the nucleus at 5 μg/mL. Lysotracker, Mitotracker, and ERtracker were detected by using excitation wavelength of 488 nm (Argon laser) and with a detection window between 510 and 530 nm. The organelle specific dyes were used according to their manuals. Cyanine dye nanoparticles were detected by using excitation wavelength of 633 nm (argon laser) and with a detection window between 680-720 nm. Cells without dyes and cyanine dye nanoparticles were used in parallel as autofluorescence controls using the corresponding excitation and detection wavelengths. Images were analyzed with LAS AF Lite (Leica).

### Organelle Colocalization Analysis by Confocal Microscopy

Cells were trypsinized and seeded in a flat-bottom μ-clear 96-well plates (Greiner Bio-One, #655087) at a density of 3,500-5,000 control and 4,000-6,000 senescent cells/well. Once the cells were attached, culture medium was changed to DMEM supplemented with 0.2% FBS and incubated with cyanine dye nanoparticles (100 μg/mL-10 μg/mL) for 12-16 h. Afterward, cells were washed 3× with PBS for 5 min. Specific organelle stains LysoTracker Green DND-26 (Cell Signaling Technology, FM8783), Mito Tracker Green (Cell Signaling Technology, FM9074), ER-Tracker™ Green (BODIPY™ FL Glibenclamide, Thermo Fisher, E34251) were used according to the manufacturers protocol. For nuclei staining, Hoechst at 0.1 μg/mL in PBS was added 10 minutes prior to analysis.

### Endocytosis Analysis by Confocal Microscopy

Cells were trypsinized and seeded in flat-bottom μ-clear 96-well plates (Greiner Bio-One, #655087) at a density of 3,500-5,000 control and 4,000-6,000 senescent cells/well. Once the cells were attached, culture medium was changed to DMEM without FBS and exposed to a number of different inhibitors for 1 h. Pitstop2 (5-15 μM, abcam ab120687), Dyngo4a (10-30 μM abcam. ab120689), Chloroquine (50 M, Tocris, 4109), Prochlorperazine dimaleate salt (15 μM, Sigma, P9178) and LY294002 (20-50 μM, Sigma, 440202). After incubation, cyanine dye nanoparticles were added in DMEM supplemented with 0.2% FBS, in a range from of 100 to 10 μg/mL for 12-16 h. The cells were washed 3x with PBS for 5 min. Specific organelle stain LysoTracker Green DND-26 (Cell Signaling Technology, FM8783) was used according to the manufacturers protocol. For nuclei staining, Hoechst at 0.1 μg/mL in PBS was added 10 min prior to analysis.

### In Vivo Studies

All mice were treated in strict accordance with the local ethical committee (University of Cambridge License Review Committee) and the UK Home Office guidelines. Tumor xenografts were established using SK-MEL-103. Cells were trypsinized, counted with a hemocytometer, and injected subcutaneously (0.5∧6 cells in a volume of 100 μL per dorsolateral flank) in 8- to 10-week-old athymic nude female mice (Hsd: Athymic Nude-Foxn1nu) purchased from Charles River. Tumor volume was measured every 2 days with a caliper and calculated as V=(α×b2)/2 where a is the longer and b is the shorter of two perpendicular diameters. Palbociclib (MCE, PD0332991) was dissolved in 50 mM sodium lactate at 10.0 mg/mL and administered by daily oral gavage at the indicated doses. 200 μL of cyanine dye nanoparticles (1 mg/mL) and 200 μL of ICG (1 mg/mL) were injected by the tail vein in DMEM (Thermfisher, no phenol red).

### Ex Vivo Imaging by IVIS

See Examples—part A. Fluorescence activity is measured as average Radiant Efficiency with the equation of [p/s/cm2/sr]/[μW/cm2].

### Photoacoustic Properties of Cyanine Dye Nanoparticles

See Examples—part A. Photoacoustic signals of the ICG monomer and cyanine dye nanoparticles were compared, after matching the peak absorbance for both to 1 AU.

### In Vivo Photoacoustic Imaging

Photoacoustic measurements were performed using a commercial PAT system (inVision256-TF; iThera Medical GmbH). Briefly, a tunable (660-1,300 nm) optical parametric oscillator, pumped by a nanosecond (ns) pulsed Nd: YAG laser, with 10 Hz repetition rate and up to 7 ns pulse duration is used for signal excitation. All mice were treated in strict accordance with the local ethical committee (University of Cambridge License Review Committee) and the UK Home Office guidelines. Tumor xenografts were established using SK-MEL-103. Cells were trypsinized, counted with a hemocytometer, and injected subcutaneously (0.5∧6 cells in a volume of 100 μL per dorsolateral flank) in 8- to 10-week-old athymic nude female mice (Hsd: Athymic Nude-Foxn1nu) purchased from Charles River. Tumor volume was measured every 2 days with a caliper and calculated as V=(α×b2)/2, where a is the longer and b is the shorter of two perpendicular diameters. Palbociclib (Pfizer Inc.) was dissolved in 50 mM sodium lactate at 10 mg/ml and administered by daily oral gavage at the indicated doses. 200 μL of cyanine dye nanoparticles (1 mg/mL) and 200 μL of ICG (1 mg/mL) were injected by the tail vein in DMEM (Thermofisher, no phenol red). Mice were anaesthetized using <3% isoflurane and placed in a custom animal holder (iThera Medical), wrapped in a thin polyethylene membrane, with ultrasound gel (Aquasonic Clear, Parker Labs) and placed in a heated water bath in the commercial PAT system (inVision256-TF; iThera Medical GmbH). Mice were imaged using the wavelengths 700, 720, 740, 760, 780, 790, 800, 820, 840, 860, 870, 880, 890, 895, 900, 905, 910, 920, and 940 nm, with an average of 10 pulses per wavelength.

PAT data analysis was performed using ViewMSOT software (v3.6.0.119; iThera Medical GmbH). Model-linear image reconstruction and linear multispectral processing were applied on data in the 700-940 nm wavelength range to retrieve the relative signal contributions of oxy-(HbO2), deoxy-(Hb) hemoglobin and the cyanine dye nanoparticles. Linear regression was performed with published spectra for ICG, oxyhemoglobin, and deoxyhemoglobin as well as collected cyanine dye nanoparticles spectrum performed in this study. Regions of interest were drawn manually over the tumor area, and then averaged over the entire tumor volume. A corresponding background ROI was drawn near the back of the mouse for each anatomical plane.

Signal to background ratio taken as the signal from the tumor ROI divided by the signal of the background ROI for each anatomical slice, this is represented as the equation

$$S BR = \sum \frac{Tumor ROI}{Background ROI} .$$

Contrast to noise ratio is defined as the sum of the differences of the ROI signal and background divided by the standard deviation of the background for each tumor slice and represented as the equation

$$CNR = \sum \frac{Tumor ROI - Background ROI}{σ Background ROIs} .$$

### Confocal Imaging of Ex Vivo Samples

For ex vivo, mice were sacrificed 6 h and 24 h post-injection and tumors placed in a cryomold containing OCT (Leica Biosystems) and frozen in dry ice for 30 min. Frozen OCT sections were washed in PBS and then imaged directly. The argon laser was used to evaluate autofluorescence of the tissue. Palbociclib untreated tumors, and Palbociclib treated tumors without cyanine dye nanoparticle injections were used as controls.

### Immunohistochemistry and Immunofluorescence

For immunohistochemistry, tumors and organs were extracted and put in 10% neutral buffered formalin (4% formaldehyde in solution), then dehydrated in 70% ethanol. These were then paraffin-embedded and sent for processing. The tissue samples were stained using Phospho-Rb (Ser807/811) and Ki-67 (D3B5). Digital image analysis was performed using HALO and the CytoNuclear v2.0.9 imaging module (Indica Labs, Albuquerque, USA).

Immunofluorescence (IF) was performed on samples previously placed in OCT and frozen. 10 μm slices were prepared using a Leica CM3050 S cryostat. Briefly, samples were washed twice with PBS, fixed in 4% PFA solution for 10 min, washed again with PBS and then permeabilized with 0.25% Triton X-100 for 15 min. Afterwards, samples were then blocked for 1 h using 2% normal donkey serum. In the same blocking solution, a 1/500 dilution of the primary antibody for Ki-67 Rabbit host (ICH-00375) from Bethyl Laboratories was added. After incubation overnight at 4° C., the slides were washed twice in PBS and incubated for 2 hours at RT with the anti-rabbit secondary antibody. Alexa Fluor® 488 AffiniPure Donkey Anti-Rabbit IgG was used (711-545-152) from Jackson Immuno Research. Slides were then mounted using fluoromount-G (0100-01) from SouthernBiotech and imaged using the Leica SP5 confocal microscope using a 20X HCX PL APO 0.5 NA dry objective or a 40X HCX PL APO 1.3 NA oil immersion objective. Images were analyzed with LAS AF Lite (Leica).

### Identification of Cellular Senescence in Mouse Embryos

Embryos were harvested at 13.5 days. Mesonephroi were surgically removed from the embryo and suspended in DMEM without phenol red (Gibco). Suspended mesonephroi were incubated with a 100 μg/mL cyanine dye nanoparticle solution overnight at 37° C. After incubation, the mesonephroi were washed 3× with PBS (Sigma) and immediately imaged using Wide Field Zeiss Axio Observer 7.

### Statistical Analysis

All analysis was performed unblinded. Statistical analyses were performed as described in the figure legend for each experiment. Statistical significance was determined Student's t tests (two-tailed) using Prism 9 software (GraphPad) as indicated. A p-value below 0.05 was considered significant and indicated with asterisk: * p<0.05, ** p<0.01, *** p <0.001, and **** p<0.00001. Where Pearson correlation was used *p<0.05, ** p<0.01, *** p<0.001, and **** p<0.00001.

### Results and Discussion

### In Vivo Detection of Senescent Burden in Chemotherapy Treated Tumors Using PAT

NanoJaggs were explored as potential contrast agents for photoacoustic imaging of senescent burden in therapy-treated tumors using live mice. Palbociclib and vehicle treated mice were treated with NanoJaggs and ICG by tail vein injection, and PAT imaging was performed 6 and 24 h after injection (FIG. 9A). To validate the senescence of Palbociclib treated tumors, β-galactosidase staining and immunohistochemistry (IHC) staining of pRB and Ki-67 were conducted on dissected tumor after in-vivo imaging. The Palbociclib treated (senescent) group showed an increased β-galactosidase activity and a loss in the nuclear staining (pRB and KI-67), indicating a reduced proliferative capacity and the implementation of senescence (FIG. 9B). The loss of Ki-67 apparent in senescent tumors was further verified with immunofluorescence (FIG. 10A). In addition, tumors treated with Palbociclib show a marked reduction in growth rate (FIG. 10B and C).

Photoacoustic images were reconstructed using ViewMSOT software (iThera Medical) which allows simultaneous imaging of hemoglobin (Hb), deoxyhemoglobin (HbO2), and NanoJagg/ICG spectra (FIG. 11A). Signal to background ratio was determined from the signal of the tumor region of interest (ROI) divided by the signal of the background produced from spectral unmixing of the NanoJagg multispectral optoacoustic tomography (MSOT) spectrum. Representative axial images of control vs senescent tumors and the quantification of the NanoJagg photoacoustic signal is shown in FIG. 9C. The signal to background ratio (SBR) was calculated at each axial plane of control and Palbociclib-treated tumors to represent the NanoJagg signal within the total tumor volume. A significantly higher SBR was observed in the senescent, Palbociclib-treated tumors (p=0.0135). This observation was further confirmed by the similar values obtained both the quantification based on contrast to-noise ratios (FIG. 11B). To confirm that the NanoJagg signal is associated with senescence, a correlation analysis was conducted using both the tumor size and growth rate (FIG. 11C). It was found that the photoacoustic NanoJagg signal strongly correlates with the arrested tumor growth rate (R²=0.82, p=0.002), but not necessarily the tumor size (R²=0.02, p=0.794). This was not seen in the case of HBO2, there was no correlation with growth rate (R²=0.097, p=0.4952, ns, FIG. 11D).

The potential of PAT imaging to be used for analysis of multiple data streams was demonstrated by analysis of non-oxygenated (Hb) and oxygenated (HbO2) hemoglobin distribution in and around tumour site. As it can be observed in FIG. 9D, NanoJaggs in non-senescent tumors colocalize with Hb and HbO2. However, in the Palbociclib-treated, senescent tumors, NanoJaggs infiltrate the tumor and are localized away from the vasculature represented by Hb and HbO2 signals.

To further confirm the presence of NanoJaggs in senescent tumours, confocal images of the tumor slices were performed (FIG. 9E). The total amount of NanoJagg-positive cells is statistically greater in senescent tumors compared to the control ones (p=0.0002). In addition, positive cells in the Palbociclib-treated tumors have significantly more NanoJagg fluorescence signal than the untreated ones (p=0.0301, FIG. 12E).

PAT was also used to monitor elimination of NanoJaggs confirming the data obtained from ex-vivo studies (FIG. 12A). Multi-spectral optoacoustic tomography (MSOT) imaging clearly shows that the NanoJaggs are eliminated through the kidneys. However, contrary to the ex-vivo images, a strong NanoJagg accumulation can also be observed in the spleen and liver. Although NanoJagg signal in the spleen and kidney reduces over time, it is not completely eliminated after 24 h (FIG. 12B).

FIGS. 13, 14 and 15 demonstrate the selective accumulation in chemotherapy induced senescent cells from metastatic mammary adenocarcinoma MDA-MB-231 (hormone independent), breast ductal carcinoma MCF-7 (hormone dependent) and pancreatic ductal adenocarcinoma (PANC-1) respectively. MDA-MB-231 and MCF-7 were induced to become senescent with 5 μM Palbociclib. Panc-1 cells were induced to become senescent with 10 μM of Gemcitabine.

FIGS. 16 Shows a cartoon of mouse embryo structures, and compares the NanoJagg staining of the mesonephros with that of X-gal. FIG. 17 shows the finer structures of the mesonephros such as the wolffian duct, and mesonephric tubules.

While specific embodiments of the invention have been described herein for the purpose of reference and illustration, various modifications will be apparent to a person skilled in the art without departing from the scope of the invention as defined by the appended claims.

1. A process for preparing a nanoparticle composition comprising cyanine dye nanoparticles dispersed in an aqueous medium, the process comprising:
(i) providing an aqueous solution of the cyanine dye;
(ii) maintaining the aqueous solution of the cyanine dye at a temperature of between 15° C. to 85° C., preferably with agitation, to allow the cyanine dye molecules to aggregate and form nanoparticles;
(iii) collecting and purifying the resultant nanoparticles present in the solution;
wherein step (iii) comprises at least one separation step wherein the cyanine dye nanoparticles formed in step (ii) are separated from the aqueous solution and re-suspended or dispersed in a different aqueous medium.

2. A process for preparing a nanoparticle composition according to claim 1, wherein the cyanine dye is selected from the group consisting of indocyanine green (ICG), IR-140, IR-820, IR-806, IR783, IR780, Cy7, and Cy7.5.

3. A process for preparing a nanoparticle composition according to claim 1 or claim 2, wherein the cyanine dye nanoparticles have a particle size of less than 1000 nm.

4. A process according to any one of the preceding claims, wherein the pH of the aqueous solution in step (i) and/or step (ii) is within the range of 2 to 9.

5. A process according to any one of the preceding claims, wherein the concentration of cyanine dye in the aqueous solution in step (i) is within the range of 0.1 mM to 10 mM.

6. A process according to any one of the preceding claims, wherein the aqueous solution of the cyanine dye is a solution of the cyanine dye in water.

7. A process according to any one of the preceding claims, wherein, in step (ii), the solution is heated to a temperature within the range of 40° C. to 85° C., optionally for 0.5 to 48 hours.

8. A process according to any one of the preceding claims, wherein, in step (ii), the process further comprises monitoring the formation of nanoparticles, optionally by monitoring the depletion of the non-aggregated cyanine dye from the aqueous solution and/or the formation of the nanoparticles.

9. A process according to any one of the preceding claims, wherein, in step (iii), the collection and purification of the cyanine dye nanoparticles comprises one, two or more or three or more centrifugation steps.

10. A process according to any one of the preceding claims, wherein, in step (iii), the collection and purification of the cyanine dye nanoparticles further comprises additional steps of dialysing the solution of cyanine dye nanoparticles and/or filtering the solution of cyanine dye nanoparticles.

11. Cyanine dye nanoparticles obtainable by/obtained by/directly obtained by a process as defined in any one of claims 1 to 10.

12. A nanoparticle composition obtainable by/obtained by/directly obtained by a process as defined in any one of claims 1 to 10.

13. A nanoparticle composition comprising cyanine dye nanoparticles of claim 11 dispersed in an aqueous medium.

14. A nanoparticle composition comprising cyanine dye nanoparticles dispersed in an aqueous medium, wherein:
(i) the composition is substantially free of cyanine dye in a non-aggregated form in the solution; and/or
(ii) the composition is stable for greater than 12 hours.

15. An imaging agent comprising cyanine dye nanoparticles according to claim 11, or a nanoparticle composition according to any of claims 12 to 14, and one or more pharmaceutically acceptable excipients.

16. Cyanine dye nanoparticles according to claim 11, or a nanoparticle composition according to any of claims 12 to 14, or an imaging agent according to claim 15, for use in:
(i) detecting senescent cells in a biological sample in vitro, ex vivo or in vivo; or
(ii) determining the senescent burden of a cell population in a biological sample in vitro, ex vivo or in vivo.

17. Use of cyanine dye nanoparticles according to claim 11, or a nanoparticle composition according to any of claims 12 to 14, or an imaging agent according to claim 15, for:
(i) detecting senescent cells in a biological sample in vitro, ex vivo or in vivo; or
(ii) determining the senescent burden of a cell population in a biological sample in vitro, ex vivo or in vivo.

18. Cyanine dye nanoparticles according to claim 11, or a nanoparticle composition according to any of claims 12 to 14, or an imaging agent according to claim 15 for use according to claim 16; or
the use of cyanine dye nanoparticles according to claim 11, or a nanoparticle composition according to any of claims 12 to 14, or an imaging agent according to claim 15, according to claim 17;
wherein the cyanine dye nanoparticles, nanoparticle composition or imaging agent is used to assess the amount of senescence in a biological sample (e.g., a cell population, organoid, 3D (e.g., 3D bioprints etc.), or tissue sample) in vitro, ex vivo or in vivo in response to one or more stimuli, optionally selected from replicative (ageing) stress, oncogene-induction, oxidative stress, therapeutic/drug treatment, radiation exposure, viral or bacterial infection, or other agents or stress factors.

19. Cyanine dye nanoparticles according to claim 11, or a nanoparticle composition according to any of claims 12 to 14, or an imaging agent according to claim 15 for use according to claim 16; or
the use of cyanine dye nanoparticles according to claim 11, or a nanoparticle composition according to any of claims 12 to 14, or an imaging agent according to claim 15, according to claim 17;
wherein the cyanine dye nanoparticles, nanoparticle composition or imaging agent is used for:
(i) detecting the presence of senescent cells or determining the senescent burden of:
a pre-malignant lesion or tumour, or
an age-related disorder (e.g., chronic disorders, including but not restricted to, cardiovascular diseases, fibrosis, neurological disorders, Type 1 & 2 diabetes, sarcopenia, osteoarthritis, osteoporosis, inflammatory diseases, COPD, infarction, aneurysm, cataracts, post-infarction tissues),
and optionally wherein a treatment (e.g. with one or more senolytics, one or more chemotherapeutic agents and/or radiation therapy) is determined based on the detection of the presence of senescent cells and/or the determination of the senescent burden;
(ii) detecting the presence of senescent cells or determining the senescent burden of a pre-malignant lesion, tumour or age-related disorder before, during or after treatment;
(iii) detecting the presence of senescent cells or determining the senescent burden of a biological sample or tissue to assess and/or monitor biological aging, developmental senescence (e.g., in physiological processes), tissue dysfunction, inflammation, viral or bacterial infection, the accumulation of senescent cells (e.g., physiological or pathological senescence);
(iv) detecting the presence of senescent cells or determining the senescent burden of a biological sample or tissue before, during or after treatment with one or more senolytics, one or more chemotherapeutic agents and/or radiation therapy.

20. Cyanine dye nanoparticles according to claim 11, or a nanoparticle composition according to any of claims 12 to 14, or an imaging agent according to claim 15 for use according to claim 16; or
the use of cyanine dye nanoparticles according to claim 11, or a nanoparticle composition according to any of claims 12 to 14, or an imaging agent according to claim 15, according to claim 17;
wherein the presence of the senescent cells is determined by magnetic resonance imaging (MRI), positron emission tomography (PET), near infrared II (NIR-II) imaging, shortwave infrared (SWIR) imaging, two-photon microscopy, fluorescence imaging or photoacoustic imaging or a combination thereof.

21. An imaging method for:
(i) detecting senescent cells in a biological sample in vitro, ex vivo or in vivo; or
(ii) determining the senescent burden of a cell population in a biological sample in vitro, ex vivo or in vivo;
the method comprising contacting cyanine dye nanoparticles according to claim 11, or a nanoparticle composition according to any of claims 12 to 14, or an imaging agent according to claim 15, to the biological sample and imaging the sample to determine the distribution of the nanoparticles and identify senescent cells in the sample.

22. An imaging method according to claim 21, wherein imaging the biological sample comprises application of at least one imaging technique selected from the group consisting of: magnetic resonance imaging (MRI), positron emission tomography (PET), near infrared II (NIR-II) imaging, shortwave infrared (SWIR) imaging, two-photon microscopy, photoacoustic imaging or fluorescent imaging or any combination thereof.

23. An imaging method according to claim 21 or 22, wherein the image is analysed to assess the amount of senescence in a biological sample (e.g., a cell population, organoid, 3D (e.g., 3D bioprints etc.), or tissue sample) in vitro, ex vivo or in vivo in response to one or more stimuli, optionally selected from replicative (ageing) stress, oncogene-induction, oxidative stress, therapeutic/drug treatment, radiation exposure, viral or bacterial infection, or other agents or stress factors.

24. An imaging method according to claim 21, 22 or 23, wherein the method is a method of:
(i) detecting the presence of senescent cells or determining the senescent burden of:
a pre-malignant lesion or tumour, or
an age-related disorder (e.g., chronic disorders, including but not restricted to, cardiovascular diseases, fibrosis, neurological disorders, Type 1 & 2 diabetes, sarcopenia, osteoarthritis, osteoporosis, inflammatory diseases, COPD, infarction, aneurysm, cataracts, post-infarction tissues, etc.),
and optionally wherein a treatment (e.g. with one or more senolytics, one or more chemotherapeutic agents and/or radiation therapy) is determined based on the detection of the presence of senescent cells and/or the determination of the senescent burden;
(ii) detecting the presence of senescent cells or determining the senescent burden of a pre-malignant lesion, tumour or age-related disorder before, during or after treatment;
(iii) detecting the presence of senescent cells or determining the senescent burden of a biological sample or tissue to assess and/or monitor biological aging, developmental senescence (e.g., in physiological processes), tissue dysfunction, inflammation, viral or bacterial infection, the accumulation of senescent cells (e.g., physiological or pathological senescence) or
(iv) detecting the presence of senescent cells or determining the senescent burden of a biological sample or tissue before, during or after treatment with one or more senolytics, one or more chemotherapeutic agents and/or radiation therapy.

25. An imaging method according to any one of claims 21 to 24, wherein the step of contacting cyanine dye nanoparticles according to claim 11, or a nanoparticle composition according to any of claims 12 to 14, or an imaging agent according to claim 15, to the biological sample comprises administering the cyanine dye nanoparticles, nanoparticle composition or imaging agent to a subject, and imaging the subject or a tissue in the subject to determine the distribution of the nanoparticles in vivo and identify any senescent cells in the subject or a tissue in the subject.
